(19) United States
(12) Patent Application Publication
(10) Pub. No.: US 2016/0024114 A1
(43) Pub. Date: Jan. 28, 2016

(54) MACROCYCLIC RIP2 KINASE INHIBITORS

(71) Applicant: ONCODESIGN S.A, Dijon Cedex (FR)

(72) Inventors: Petra Marcella, Francoise Blom, Destelbergen (BE); Jan Marie Cyriel Jozef Hoflack, Westmalle (BE); Pascal Andre, Rene Benderitter, St. Apollinaire (FR)

(73) Assignee: ONCODESIGN S.A., Dijon Cedex (FR)

(21) Appl. No.: 14/776,889

(22) PCT Filed: Mar. 14, 2014
(86) PCT No.: PCT/EP2014/055139

§ 371 Date: Sep. 15, 2015

(30) Foreign Application Priority Data
Mar. 15, 2013 (EP) PCT/EP2013/066732
Mar. 15, 2013 (GB) 1304714.7

(51) Int. Cl. C07D 498/22; C07D 498/16

(52) U.S. Cl. CPC C07D 498/22; C07D 498/16

(57) ABSTRACT

The present invention relates to macrocyclic compounds and compositions containing said compounds acting as kinase inhibitors, in particular as inhibitors of RIP2 and/or mutants thereof, for use in the diagnosis, prevention and/or treatment of RIP2-kinase associated diseases. Moreover, the present invention provides methods of using said compounds, for instance as a medicine or diagnostic agent.

### FIELD OF THE INVENTION

The present invention relates to macrocyclic compounds and compositions containing said compounds acting as kinase inhibitors, in particular as inhibitors of RIP2, and/or mutants thereof, for use in the diagnosis, prevention and/or treatment of RIP2-kinase associated diseases. Moreover, the present invention provides methods of using said compounds, for instance as a medicine or diagnostic agent.

### BACKGROUND OF THE INVENTION

Protein kinases constitute a large family of structurally related enzymes that are responsible for the control of a wide variety of signal transduction processes in the cell. They have been shown to be key regulators in most cellular functions including proliferation, cell metabolism, cell survival, apoptosis, DNA damage repair, cell motility . . . . Uncontrolled signalling due to defective control of protein phosphorylation has been implicated in a number of diseases, including, for example, cancer, inflammation, allergies, immune diseases, CNS disorders, angiogenesis . . . .

Amongst the families of protein kinases, one particular example is the Receptor-Interacting Serine/Threonine Kinases including RIP2. RIP2 (Receptor-Interacting Protein 2) is also referred to as Card-Containing Ice-Associated Kinase (CARDIAK), CARD3 (C-terminal CAspase-Recruitment Domain 3), Receptor-Interacting Protein Kinase 2 (RIPK2), or Rip-Like Interacting Clarp Kinase (RICK). RIP2 kinase is composed of an N-terminal kinase domain and a C-terminal caspase-recruitment domain (CARD) linked via an intermediate (IM) region (Curr. Med. Chem. (2005) 4, 35-42)). The CARD domain of RIP2 kinase mediates interaction with other CARD-containing proteins, such as the Nucleotide Oligomerization Domain Proteins, NOD1 and NOD2 (J. Biol. Chem. (2000) 275, 27823-27831 and EMBO reports (2001) 2, 736-742). NOD1 and NOD2 are cytoplasmic receptors which are activated by specific bacterial peptidoglycan motifs and play a key role in innate immune surveillance. Upon intracellular bacterial exposure, NOD1 or NOD2 binds to the protein kinase RIP2 to coordinate NF-κB (nuclear factor κ B)-mediated cytokine responses. Once associated with NOD1/2, RIP2 undergoes autophosphorylation on Tyr 474 (Y474), and acts as a molecular scaffold to bring together other kinases (TAK1, IKKα/β/γ) involved in NF-κB and MAPK activation (Nature Reviews Immunology (2006) 6, 9-20).

Both NOD1/2 and RIP2 are NF-κB regulated genes, and as such, their activation causes a positive feedback loop in which activation of NOD1/2:RIP2 stimulates further activation and further inflammation. Additionally, NOD1/2 and RIP2 expression are stimulated by a variety of mediators of inflammation, including TNF (Tumor Necrosis Factor) and IFN (Interferon). In addition to NF-κB pathway activation, the NOD1/2:RIP2 complex stimulates autophagy, bacteriocidal activity, MHC Class II presentation and MAPK (Mitogen-Activated Protein Kinase) activation. Overall, this pathway modulates the innate immune system to help tailor the adaptive immune response to eradicate the offending pathogen.

Dysregulation of RIP2-dependent signaling has been linked to autoinflammatory diseases. Patients with loss-of-function NOD2 alleles are prone to the development of Crohn's disease, an inflammatory disorder of the gastrointestinal tract (Am. J. Hum. Genet. (2002) 70, 845-857 and Microbes and Infection (2009) 11, 912-918). In contrast, gain-of-function NOD2 mutations have been genetically linked to other inflammatory diseases, such as Blau Syndrome/Early Onset Sarcoidosis (EOS), a pediatric granulomateous disease characterized by uveitis, dermatitis, and arthritis (Nature Genetics (2001) 29, 19-20 and Current Rheumatology Reports (2005) 7, 427-433). Mutations in NOD1 have been associated with asthma (Hum. Mol. Genet. (2005) 14, 935-941), and early-onset and extra-intestinal inflammatory bowel disease (Hum. Mol. Genet. (2005) 14, 1245-1250). Genetic and functional studies have also suggested a role for RIP2-dependent signaling in a variety of other granulomateous disorders, such as sarcoidosis (Journal of Clinical Immunology (2009) 29, 78-89) and Wegner's Granulomatosis (Diagnostic Pathology (2009) 4, 23).

The fact that both loss-of-function polymorphisms and gain-of-function mutations cause inflammatory diseases is likely due to the fact that NOD2 functions as a rheostat to help maintain normal immunologic homeostasis. Lack of coordination between inflammatory signaling pathways influences the development of inflammatory disorders, and the NOD1/2:RIP2 activation equilibrium is central to this coordination. Treatments for Crohn's disease and sarcoidosis currently rely on broad, non-specific immunologic inhibition (e.g., corticosteroids) or on specific cytokine inhibition (e.g., anti-TNF therapies) with significant costs and side effects. Treatment is less than ideal, however, because not all agents are equally efficacious, the diseases occur over long time frames, and not all agents remain efficacious in the same patient. The RIP2 Y474 autophosphorylation event has been shown to be necessary for effective NOD2 signaling and does not occur in the presence of the most common loss-of-function Crohn's disease-associated NOD2 allele. This autophosphorylation is inhibited by non highly selective kinase inhibitors, gefitinib and erlotinib, suggesting that RIP2's tyrosine kinase activity could be targeted specifically in the treatment of inflammatory diseases (Genes Dev. (2010) 1, 2666-77). Several clinical cases were reported about gefitinib or erlotinib treatment being efficient to clear psoriasis or reduce arthritic symptoms or insulin-resistant type 2 diabetes associated with metabolic syndrome (The Oncologist (2013) 18: e3-e5). In mouse established models of chronic inflammatory bowel diseases, inhibition of RIP2 activity by the small molecule SB203580 is efficacious to reduce induced-colitis (J Biol Chem. (2005) 15, 14981-14988.). None of these small molecules however, primarily and selectively targets RIP2. It was therefore an object of the present invention to provide a potent, selective, small molecule inhibitor of RIP2 kinase activity which can block specifically RIP2-dependent pro-inflammatory signaling and thereby provides a therapeutic benefit in autoinflammatory diseases characterized in increased and/or dysregulated RIP2 kinase activity.

We have now found that the macrocyclic pyrazolopyrimidines and imidazopyridazines and pharmaceutically acceptable compositions according to this invention are useful for the treatment of inflammatory disorders, in particular Crohn's disease, bowel disease, Sarcoidosis, psoriasis, rheumatoid arthritis, asthma and insulin-resistant type 2 diabetes, ulcerative colitis, lupus, uveitis, blau syndrome, granulomatous inflammation, in particular behget's disease, multiple sclerosis, and disease associated with RIP2 kinase activity (i.e. RIP2-kinase associated diseases).

### SUMMARY OF THE INVENTION

We have surprisingly found that the macrocyclic compounds described herein act as RIP2 kinase inhibitors, and are thus very useful in the diagnosis, prevention and/or treatment of RIP2-kinase associated diseases.

In a first objective the present invention provides a compound of Formula I or a stereoisomer, tautomer, racemic, metabolite, pro- or predrug, salt, hydrate, N-oxide form, or solvate thereof,

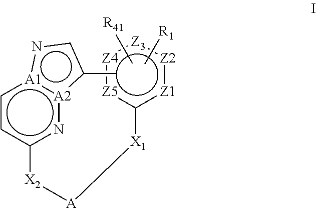

Wherein
A₁ and A₂ are selected from C and N; wherein when A₁ is C, then A₂ is N; and wherein when A₂ is C, then A₁ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl -Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H —C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a first embodiment the present invention provides a compound of Formula I or a stereoisomer, tautomer, racemic, metabolite, pro- or predrug, salt, hydrate, N-oxide form, or solvate thereof, wherein
A₁ is C and A₂ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl -Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H —C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a further embodiment, the present invention provides a compound of Formula I or a stereoisomer, tautomer, racemic, metabolite, pro- or predrug, salt, hydrate, N-oxide form, or solvate thereof, wherein
A₁ is N and A₂ is C
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl -Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;

R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alky, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alky, —S—C₁₋₆alky, —NR₄₆R₄₇, —C₃₋₆cycloalkyl, —Ar₉ and -Het₈;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl-Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a further embodiment, the present invention provides a compound of Formula I or a stereoisomer, tautomer, racemic, metabolite, pro- or predrug, salt, hydrate, N-oxide form, or solvate thereof, wherein
A₁ and A₂ are selected from C and N; wherein when A₁ is C, then A₂ is N; and wherein when A₂ is C, then A₁ is N;
R₁ and R₄₁ are each independently selected from —H, -halo, —C₁₋₆alkyl, —(C═O)—R₄, and —CN; wherein each of said —C₁₋₆alkyl is optionally and independently substituted with from 1 to 3 substituents selected from —O—C₁₋₆alkyl;
R₂ is selected from —H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;

R₆ is selected from —C₁₋₆alkyl, —(C═O)—C₁₋₆alkyl, —(C═O)—C₃₋₆cycloalkyl, -Het₆, and —C₃₋₆cycloalkyl;
wherein each of said —C₁₋₆alkyl is optionally and independently substituted with from 1 to 3 substituents selected from —O—C₁₋₆alkyl and -Het₆;
and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from —H, and —C₁₋₆alkyl;
A is selected from —(CH₂)ₙ—Y—(CH₂)ₘ, —NR₆—, and —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl; Y is —NR₄₃—;
Het₆ is a 4- to 10-membered heterocycle having from 1 to 3 heteroatoms selected from O, N and S;

Z₁, Z₂, Z₃, Z₄ and Z₅ are each independently selected from C and N; and m and n are each independently 1, 2, 3, or 4;
for use in the diagnosis, prevention and/or treatment of a RIP2-kinase associated disease.

In a further embodiment, the present invention provides a compound of Formula I or a stereoisomer, tautomer, racemic, metabolite, pro- or predrug, salt, hydrate, N-oxide form, or solvate thereof, wherein
A₁ is C and A₂ is N;

R₁ and R₄₁ are each independently selected from —H, -halo, —C₁₋₆alkyl, —(C═O)—R₄, and —CN;
wherein each of said —C₁₋₆alkyl is optionally and independently substituted with from 1 to 3 substituents selected from —O—C₁₋₆alkyl;
R₂ is selected from H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;
R₆ is selected from —C₁₋₆alkyl, —(C═O)—C₁₋₆alkyl, —(C═O)—C₃₋₆cycloalkyl, -Het₆, and —C₃₋₆cycloalkyl;
   wherein each of said —C₁₋₆alkyl is optionally and independently substituted with from 1 to 3 substituents selected from —O—C₁₋₆alkyl and -Het₆;
   and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from H, and —C₁₋₆alkyl;
R₄₃ is selected from —H, and —C₁₋₆alkyl;
A is selected from —(CH₂)ₙ—Y—(CH₂)ₘ—, —NR₆—, and —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is —NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a further embodiment, the present invention provides a compound of Formula I or a stereoisomer, tautomer, racemic, metabolite, pro- or predrug, salt, hydrate, N-oxide form, or solvate thereof, wherein
A₁ is N and A₂ is 0;
R₁ and R₄₁ are each independently selected from —H, -halo, —C₁₋₆alkyl, —(C═O)—R₄, and —CN;
   wherein each of said —C₁₋₆alkyl is optionally and independently substituted with from 1 to 3 substituents selected from —O—C₁₋₆alkyl;
R₂ is selected from H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;
R₆ is selected from —C₁₋₆alkyl, —(C═O)—C₁₋₆alkyl, —(C═O)—C₃₋₆cycloalkyl, -Het₆, and —C₃₋₆cycloalkyl;
   wherein each of said —C₁₋₆alkyl is optionally and independently substituted with from 1 to 3 substituents selected from —O—C₁₋₆alkyl and -Het₆;
   and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from H, and —C₁₋₆alkyl;
R₄₃ is selected from H, and —C₁₋₆alkyl;
A is selected from —(CH₂)ₙ—Y—(CH₂)ₘ—, and —NR₆—, —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is —NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a further aspect, the present invention provides a compound according to the present invention for use in the diagnosis, prevention and/or treatment of a RIP2-kinase associated disease; wherein the pyrazolopyrimidine or the imidazopyridazine moiety is linked to the aryl or heteroaryl moiety at position Z₄ or Z₅, in accordance with the numbering as provided in Formula I.

In yet a further aspect, the present invention provides a compound according to the present invention for use in the diagnosis, prevention and/or treatment of a RIP2-kinase associated disease; wherein R₁ is linked to the aryl or heteroaryl moiety at position Z₁, Z₂ or Z₃, in accordance with the numbering as provided in Formula I.

In yet a further aspect, the present invention provides a compound selected from the list comprising:

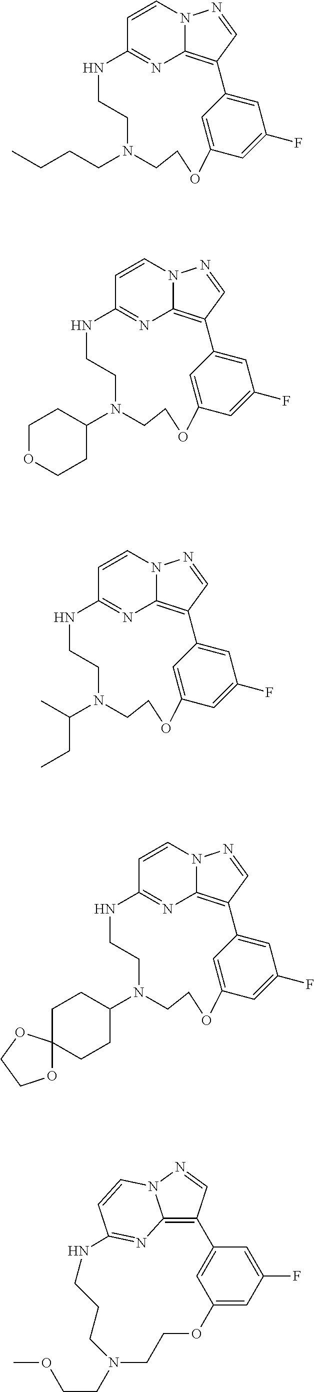 [SMILES: CCCCN1CCNc2ccn3ncc(c3n2)-c2cc(F)cc(c2)OCC1 ; Fc1cc2cc(c1)-c1cnn3ccc(nc13)NCCN(C1CCOCC1)CCO2 ; CCC(C)N1CCNc2ccn3ncc(c3n2)-c2cc(F)cc(c2)OCC1 ; Fc1cc2cc(c1)-c1cnn3ccc(nc13)NCCN(C1CCC3(CC1)OCCO3)CCO2 ; COCCN1CCCNc2ccn3ncc(c3n2)-c2cc(F)cc(c2)OCC1]

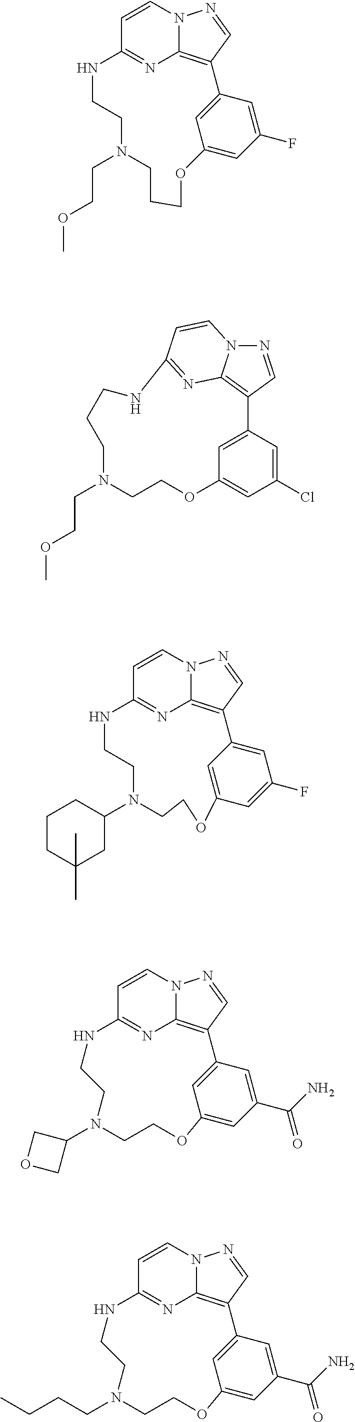

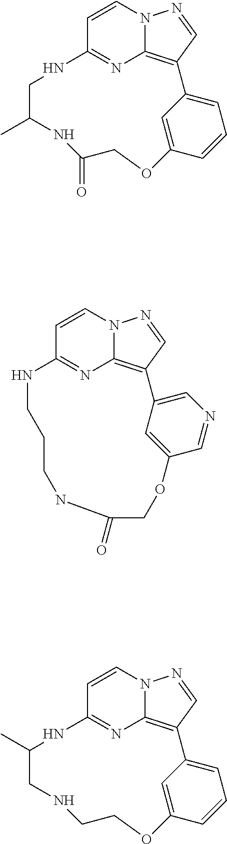 [SMILES: CC1CNc2ccn3ncc(c3n2)-c2cccc(c2)OCC(=O)N1 ; O=C1COc2cncc(c2)-c2cnn3ccc(nc23)NCCCN1 ; CC1CNCCOc2cccc(c2)-c2cnn3ccc(nc23)N1]

In a particular embodiment, the RIP2-kinase associated disease is an inflammatory disorder, more in particular Crohn's disease, bowel disease, Sarcoidosis, psoriasis, rheumatoid arthritis, asthma, ulcerative colitis, lupus, uveitis, blau syndrome, granulomatous inflammation, in particular behget's disease, multiple sclerosis and insulin-resistant type 2 diabetes.

The present invention further provides a pharmaceutical composition for use in the prevention and/or treatment of a RIP2-kinase associated disease comprising a compound according to this invention.

Furthermore, the present invention provides the use of a compound or composition according to this invention, suitable for inhibiting the activity of a kinase; in particular a RIP2 kinase; or for the diagnosis, prevention and/or treatment of a RIP2-kinase associated disease.

Finally, the present invention provides a method for prevention and/or treatment of a RIP2-kinase associated disease; said method comprising administering to a subject in need thereof a compound or a composition according to the present invention.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Unless a context dictates otherwise, asterisks are used herein to indicate the point at which a mono- or bivalent radical depicted is connected to the structure to which it relates and of which the radical forms part.

As already mentioned hereinbefore, in a first aspect the present invention provides a compound of Formula I or a stereoisomer, tautomer, racemic, metabolite, pro- or predrug, salt, hydrate, N-oxide form, or solvate thereof,

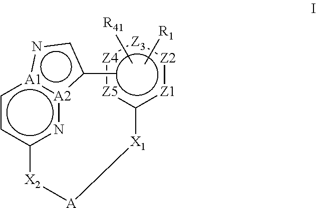

Wherein
A₁ and A₂ are selected from C and N; wherein when A₁ is C, then A₂ is N; and wherein when A₂ is C, then A₁ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(O═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇1R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)−C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C—O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—R₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O )—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₆;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₈—, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

Unless indicated otherwise, all of the above radicals can be read both ways. For example, when A is —(C═O)—NR₆—, the —(C═O)— may be attached to X₂ and —NR₅— attached to X₁. Alternatively, the —(C═O)— may be attached to X₁ and —NR₅— attached to X₁. What is called “left part” of a radical is for example when A is —(C═O)—NR₅—, —(C═O)—, and the “right part” is —NR₅—.

Preferably, A is such as the left part of the possible values of A (i.e. in particular —(C═N) from —(C═N)—R₄₃, —(C═O) from —(C═O)—NR₅, —(C═S) from —(C═S)—NR₅, —SO₂ from —SO₂—NR₅—, etc) is attached to X₁. Alternatively, A is such as the right part of the possible values of A (i.e. in particular (R₄₉)— from —(C═N)R₄₉, (NR₅)— from —(C═O)—NR₅, —NR₅ from —(C═S)—NR₅, —NR_{S}— from —SO₂—NR₅—, etc) is attached to X₁.

Preferably, X₁ is such as the left part of the possible values of X₁ (i.e. in particular —O from —O—C₁₋₆alkyl, —S from —S—C₁₋₆alkyl, —NR₃ from —NR₃—(C═O) and —NR₃—C₁₋₆alkyl, —SO₂ from —SO₂—NR₃, etc) is attached to the Z₁-Z₅ aryl or heteroaryl moiety. Alternatively, X₁ is such as the right part of the possible values of X₁ (i.e. in particular (C₁₋₆alkyl)- from —O—C₁₋₆alkyl, —S—C₁₋₆alkyl and —NR₃—C₁₋₆alkyl, —(C═O) from —NR₃—(C═O), (NR₃)— from —SO₂—NR₃, etc) is attached to the Z₁-Z₅ aryl or heteroaryl moiety.

Preferably, X₂ is such as the left part of the possible values of X₂ (i.e. in particular —O from —O—C₁₋₆alkyl, —S from —S—C₁₋₆alkyl, —(C═O) from —(C═O)—NR₂, —NR₂ from —NR₂—C₁₋₆alkyl, —SO₂ from —SO₂—NR₂, etc) is attached to the pyrazolopyrimidine moiety. Alternatively, X₂ is such as the right part of the possible values of X₂ (i.e. in particular (C₁₋₆alkyl)- from —O—C₁₋₆alkyl, —S—C₁₋₆alkyl and —NR₂—C_{1- 6}alkyl, (NR₂)— from —(C═O)—NR₂ and —SO₂—NR₂, etc) is attached to the pyrazolopyrimidine moiety. The same principle applies to all the radicals of the invention unless specified otherwise.

When describing the compounds of the invention, the terms used are to be construed in accordance with the following definitions, unless a context dictates otherwise:

The term “alkyl” by itself or as part of another substituent refers to fully saturated hydrocarbon radicals. Generally, alkyl groups of this invention comprise from 1 to 6 carbon atoms. Alkyl groups may be linear or branched and may be substituted as indicated herein. When a subscript is used herein following a carbon atom, the subscript refers to the number of carbon atoms that the named group may contain. Thus, for example, C₁₋₆alkyl means an alkyl of one to six carbon atoms. Examples of alkyl groups are methyl, ethyl, n-propyl, i-propyl, butyl, and its isomers (e.g. n-butyl, i-butyl and t-butyl); pentyl and its isomers, hexyl and its isomers. C₁-C₆ alkyl includes all linear, branched, or cyclic alkyl groups with between 1 and 6 carbon atoms, and thus includes methyl, ethyl, n-propyl, i-propyl, butyl and its isomers (e.g. n-butyl, i-butyl and t-butyl); pentyl and its isomers, hexyl and its isomers, cyclopropyl, cyclobutyl, cyclopentyl, and cyclohexyl.

The term “optionally substituted alkyl” refers to an alkyl group optionally substituted with one or more substituents (for example 1 to 3 substituents, for example 1, 2 or 3 substituents or 1 to 2 substituents) at any available point of attachment. Non-limiting examples of such substituents include -halo, —OH, primary and secondary amides, —O—C₁₋₆alkyl, —S—C₁₋₆alkyl, heteroaryl, aryl, and the like.

The term “cycloalkyl” by itself or as part of another substituent is a cyclic alkyl group, that is to say, a monovalent, saturated, or unsaturated hydrocarbyl group having a cyclic structure. Cycloalkyl includes all saturated or partially saturated (containing 1 or 2 double bonds) hydrocarbon groups having a cyclic structure. Cycloalkyl groups may comprise 3 or more carbon atoms in the ring and generally, according to this invention comprise from 3 to 6 atoms. Examples of cycloalkyl groups include but are not limited to cyclopropyl, cyclobutyl, cyclopentyl, cyclohexyl.

Cycloalkyl as referred herein also includes substituted cycloalkyl groups, wherein such groups may be substituted once or more, and preferably once, twice or thrice. Substituents may be selected from —C₁₋₆alkyl and those defined above for substituted alkyl.

Where alkyl groups as defined are divalent, i.e., with two single bonds for attachment to two other groups, they are termed “alkylene” groups. Non-limiting examples of alkylene groups includes methylene, ethylene, methylmethylene, trimethylene, propylene, tetramethylene, ethylethylene, 1,2-dimethylethylene, pentamethylene and hexamethylene.

Generally, alkylene groups of this invention preferably comprise the same number of carbon atoms as their alkyl counterparts. Where an alkylene or cycloalkylene biradical is present, connectivity to the molecular structure of which it forms part may be through a common carbon atom or different carbon atom. To illustrate this applying the asterisk nomenclature of this invention, a C₃ alkylene group may be for example *—CH₂CH₂CH₂—*, *—CH(—CH₂CH₃)—*, or *—CH₂CH(—CH₃)—*. Likewise a C₃ cycloalkylene group may be

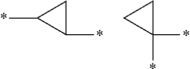

The terms “heterocycle” as used herein by itself or as part of another group refer to non-aromatic, fully saturated or partially unsaturated cyclic groups (for example, 3 to 6 membered monocyclic ring systems, or 8-10 membered bicyclic rings) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms. An optionally substituted heterocyclic refers to a heterocyclic having optionally one or more substituents (for example 1 to 4 substituents, or for example 1, 2, 3 or 4), selected from those defined above for substituted alkyl.

Exemplary heterocyclic groups include piperidinyl, azetidinyl, imidazolinyl, imidazolidinyl, isoxazolinyl, oxazolidinyl, isoxazolidinyl, thiazolidinyl, isothiazolidinyl, piperidyl, succinimidyl, 3H-indolyl, isoindolinyl, chromenyl, isochromanyl, xanthenyl, 2H-pyrrolyl, 1-pyrrolinyl, 2-pyrrolinyl, 3-pyrrolinyl, pyrrolidinyl, 4H-quinolizinyl, 4aH-carbazolyl, 2-oxopiperazinyl, piperazinyl, homopiperazinyl, 2-pyrazolinyl, 3-pyrazolinyl, pyranyl, dihydro-2H-pyranyl, 4H-pyranyl, 3,4-dihydro-2H-pyranyl, phthalazinyl, oxetanyl, thietanyl, 3-dioxolanyl, 1,3-dioxanyl, 2,5-dioximidazolidinyl, 2,2,4-piperidonyl, 2-oxopiperidinyl, 2-oxopyrrolodinyl, 2-oxoazepinyl, indolinyl, tetrahydropyranyl, tetrahydrofuranyl, tetrehydrothienyl, tetrahydroquinolinyl, tetrahydroisoquinolinyl, thiomorpholinyl, thiomorpholinyl sulfoxide, thiomorpholinyl sulfone, 1,3-dioxolanyl, 1,4-oxathianyl, 1,4-dithianyl, 1,3,5-trioxanyl , 6H-1,2,5-thiadiazinyl, 2H-1,5,2-dithiazinyl , 2H-oxocinyl, 1H-pyrrolizinyl , tetrahydro-1,1-dioxothienyl , N-formylpiperazinyl, and morpholinyl; in particular pyrrolidinyl, imidazolidinyl, pyrazolidinyl, piperidinyl, dioxolanyl, dioxanyl, morpholinyl, thiomorpholinyl, piperazinyl, thiazolidinyl, tetrahydropyranyl, and tetrahydrofuranyl. 8-10 membered heterocyclic groups are also meant to include spiro-groups, which are bicyclic compounds with both rings connected through a single atom, such as for example spiro[4.5]decane, which is a spiro compound consisting of a cyclohexane ring and a cyclopentane ring.

The term “aryl” as used herein refers to a polyunsaturated, aromatic hydrocarbyl group having from 5-10 atoms. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7-, or 8-azulenyl, 1- or 2-naphthyl, 1-, 2-, or 3-indenyl, 1-, 2-, or 9-anthryl, 1-2-, 3-, 4-, or 5-acenaphtylenyl, 3-, 4-, or 5-acenaphtenyl, 1-, 2-, 3-, 4-, or 10-phenanthryl, 1- or 2-pentalenyl, 1,2-, 3-, or 4-fluorenyl, 4- or 5-indanyl, 5-, 6-, 7-, or 8-tetrahydronaphthyl, 1,2,3,4-tetrahydronaphthyl, 1,4-dihydronaphthyl, dibenzo[a,d]cylcoheptenyl, and 1-, 2-, 3-, 4-, or 5-pyrenyl; in particular phenyl.

The aryl ring can optionally be substituted by one or more substituents. An “optionally substituted aryl” refers to an aryl having optionally one or more substituents (for example 1 to 5 substituents, for example 1, 2, 3 or 4) at any available point of attachment, selected from those defined above for substituted alkyl.

Where a carbon atom in an aryl group is replaced with a heteroatom, the resultant ring is referred to herein as a heteroaryl ring.

The term “heteroaryl” as used herein by itself or as part of another group refers but is not limited to 5 to 10 carbon-atom aromatic rings in which one or more carbon atoms can be replaced by oxygen, nitrogen or sulfur atoms. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl, 7-azaindolyl, 6-azaindolyl, 5-azaindolyl, 4-azaindolyl.

An “optionally substituted heteroaryl” refers to a heteroaryl having optionally one or more substituents (for example 1 to 4 substituents, for example 1, 2, 3 or 4), selected from those defined above for substituted alkyl.

The term “halo” or “halogen” as a group or part of a group is generic for fluoro, chloro, bromo, or iodo, as well as any suitable isotope thereof.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic and/or diagnostic agent.

Where groups may be optionally substituted, such groups may be substituted once or more, and preferably once, twice or thrice. Substituents may be selected from, those defined above for substituted alkyl.

As used herein the terms such as “alkyl, aryl, or cycloalkyl, each being optionally substituted with” or “alkyl, aryl, or cycloalkyl, optionally substituted with” refers to optionally substituted alkyl, optionally substituted aryl and optionally substituted cycloalkyl.

More generally, from the above, it will be clear to the skilled person that the compounds of the invention may exist in the form of different isomers and/or tautomers, including but not limited to geometrical isomers, conformational isomers, E/Z-isomers, stereochemical isomers (i.e. enantiomers and diastereoisomers) and isomers that correspond to the presence of the same substituents on different positions of the rings present in the compounds of the invention. All such possible isomers, tautomers and mixtures thereof are included within the scope of the invention.

In addition, the invention includes isotopically-labelled compounds and salts, which are identical to compounds of formula (I), but for the fact that one or more atoms are replaced by an atom having an atomic mass or mass number different from the atomic mass or mass number most commonly found in nature. Examples of isotopes that can be incorporated into compounds of formula (I) are isotopes of hydrogen, carbon, nitrogen, fluorine, such as ³H, ¹¹C, ¹³N, ¹⁴C, ¹⁵O and ¹⁸F. Such isotopically-labelled compounds of formula (I) are useful in drug and/or substrate tissue distribution assays. For example ¹¹O and ¹⁸F isotopes are particularly useful in PET (Positron Emission Tomography). PET is useful as a diagnostic or treatment follow-up tool that can be applied in a translational manner in a preclinical and clinical setting. It also has applications in PK determination of compounds, including biodistribution. Isotopically labeled compounds of formula (I) can generally be prepared by carrying out the procedures disclosed below, by substituting a readily available non-isotopically labeled reagent with an isotopically labeled reagent.

Whenever used in the present invention the term “compounds of the invention” or a similar term is meant to include the compounds of general Formula I and any subgroup thereof. This term also refers to the compounds as depicted in Table 1, their derivatives, N-oxides, salts, solvates, hydrates, stereoisomeric forms, racemic mixtures, tautomeric forms, optical isomers, analogues, pro-drugs, esters, and metabolites, as well as their quaternized nitrogen analogues. The N-oxide forms of said compounds are meant to comprise compounds wherein one or several nitrogen atoms are oxidized to the so-called N-oxide.

As used in the specification and the appended claims, the singular forms “a”, “an”, and “the” include plural referents unless the context clearly dictates otherwise. By way of example, “a compound” means one compound or more than one compound.

The terms described above and others used in the specification are well understood to those in the art.

In a particular embodiment, the present invention provides compounds of Formula I or a stereoisomer, tautomer, racemic, metabolite, pro- or predrug, salt, hydrate, N-oxide form, or solvate thereof; for use in the diagnosis prevention and/or treatment of a RIP2-kinase associated disease; wherein one or more of the following applies
A₁ and A₂ are selected from C and N; wherein when A₁ is C, then A₂ is N; and wherein when A₂ is C, then A₁ is N;
R₁ and R₄₁ are each independently selected from —H, -halo, —OH, —C₁₋₆alkyl, —O—C₁₋₆alkyl, —S—C₁₋₆alkyl, —NR₃R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆ alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)-Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₃R₃₀, —(C═S)—NR₂₃R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, 'S—C₁₋₆alkyl, -Het₁₂, —Ar₁₁, and —NR₅₃R₅₄, —(O═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅ , —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —S—C₁₋₆alkylm —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₉, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₆—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(O═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₆, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₆, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;

In particular, X₁, and X₂ as used herein, represent biradicals, which taken together with the radicals to which they are attached form a macrocyclic pyrazolopyrimidine compound. Said biradicals may be present in either of both directions in the macrocyclic pyrazolopyrimidine, but are preferably present in the direction as described below:

Referring to formula I:
X₁ is selected from the list comprising *—C₁₋₆alkyl-, *—O—C₁₋₆alkyl-, *—S—C₁₋₆alkyl-, *—(C═O)—, —NR₃—(C═O)—, *—C₁₋₆alkyl-NR₃—, *—NR₃—, *—(C═O)—, *—NR₃—(C═O)—NR₄₈—, *—NR₃—C₁₋₆alkyl-, *—NR₃—SO₂—, *—NR₃—(C═O)—C₁₋₆alkyl-, *—(C═O)—NR₃—C₁₋₆alkyl-, *—O—C₁₋₆alkyl—O—C₁₋₆alkyl- and *—C₁₋₆alkyl-NR₃—C₁₋₆alkyl-; wherein said biradical is preferably attached to the aryl or heteroaryl moiety via *;
X₂ is selected from the list comprising *—C₁₋₆alkyl-, *—O—C₁₋₆alkyl-, *—S—C₁₋₆alkyl-, *—(C═O)—, *—NR₂—(C═O)—, *—C₁₋₆alkyl-NR₂—, *—NR₂—, *—(C═O)—, *—NR₂—(C═O)—NR₅₀—, *—NR₂—C₁₋₆alkyl-, *—NR₂—SO₂—, *—NR₂—(C═O)—C₁₋₆alkyl-, *—(C═O)—NR₂—C₁₋₆alkyl-, *—O—C₁₋₆alkyl-O—C₁₋₆alkyl- and *—C₁₋₆alkyl-NR₂—C₁₋₆alkyl-; wherein said biradical is preferably attached to the pyrazolopyrimidine moiety via *;

In a preferred embodiment, the present invention provides compounds of formula I or a stereoisomer, tautomer, racemic, metabolite, pro- or predrug, salt, hydrate, N-oxide form, or solvate thereof, for use in the diagnosis, prevention and/or treatment of a RIP2-kinase associated disease wherein
A₁ is C and A₂ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)-Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C—S)—O—C₁₋₆alkyl, —(C═O)—NR₂₉R₃₀, —(C═S)—NR₂₉R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(O═S)-Het₆, —(C═S)—Ar₆, —(C═S)—C₃₋₆Cycloalkyl, —(C═S)—NR₃₁R₃₂, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅ , —NR₃₄—(C═O)—O—R₃₅, —NR₃₄—(C═S)—O—R₃₅, —O—(C═O)—NR₄₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₆;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,-, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4;
for use in the diagnosis, prevention and/or treatment of a RIP2-kinase associated disease.

In a further embodiment, the present invention provides a compound of Formula I or a stereoisomer, tautomer, racemic, metabolite, pro- or predrug, salt, hydrate, N-oxide form, or solvate thereof, for use in the diagnosis, prevention and/or treatment of a RIP2-kinase associated disease wherein
A₁ is N and A₂ is C
R₁ and R₄₁ are each independently selected from -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)-C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O—NR₂₉R₃₀, —(C═S)—NR₂₉R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
   wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅R₂₆, —(C═S)—NR₂₅R₂₆, and —NR₃₃(C═S)—NR₂₅R₂₆; and
   wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;

R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —S—C₁₋₆alkyl-NR₂—C₁₋₆alkyl-; wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁ₗ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₉; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said -C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4.

In a further embodiment, the present invention provides a compound of Formula I or a stereoisomer, tautomer, racemic, metabolite, pro- or predrug, salt, hydrate, N-oxide form, or solvate thereof, for use in the diagnosis, prevention and/or treatment of a RIP2-kinase associated disease wherein
A₁ and A₂ are selected from C and N; wherein when A₁ is C, then A₂ is N; and wherein when A₂ is C, then A₁ is N;
R₁ and R₄₁ are each independently selected from —H, -halo, —C₁₋₆alkyl, —(C═O)—R₄, and —CN;
   wherein each of said —C₁₋₆alkyl is optionally and independently substituted with from 1 to 3 substituents selected from —O—C₁₋₆alkyl;
R₂ is selected from —H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;
R₆ is selected from —C₁₋₆alkyl, —(C═O)—C₁₋₆alkyl, —(C═O)—C₃₋₆cycloalkyl, -Het₆, and —C₃₋₆cycloalkyl;
   wherein each of said —C₁₋₆alkyl is optionally and independently substituted with from 1 to 3 substituents selected from —O—C₁₋₆alkyl and -Het₆;
   and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from —H, and —C₁₋₆alkyl;
A is selected from —(CH₂)ₙ—Y—(CH₂)ₘ, and —NR₆—, —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4;

In a further embodiment, the present invention provides a compound of Formula I or a stereoisomer, tautomer, racemic, metabolite, pro- or predrug, salt, hydrate, N-oxide form, or solvate thereof, for use in the diagnosis, prevention and/or treatment of a RIP2-kinase associated disease wherein
A₁ is C and A₂ is N;
R₁ and R₄₁ are each independently selected from —H, -halo, —C₁₋₆alkyl, —(C═O)—R₄, and —CN;
   wherein each of said —C₁₋₆alkyl is optionally and independently substituted with from 1 to 3 substituents selected from —O—C₁₋₆alkyl;
R₂ is selected from H, and —C₁₋₆alkyl; wherein each of said —C₁₋₆alkyl is optionally and independently substituted with —NR₁₃R₁₄;
R₃ is selected from H, and —C₁₋₆alkyl; wherein each of said —C₁₋₆alkyl is optionally and independently substituted with —NR₁₅R₁₆;
R₄ is —NR₁₇R₁₈;
R₅ is H;
R₆ is selected from —C₁₋₆alkyl, —(C═O)—C₁₋₆alkyl, —(C═O)—C₃₋₆cycloalkyl, -Het₆, and —C₃₋₆cycloalkyl;
   wherein each of said —C₁₋₆alkyl is optionally and independently substituted with from 1 to 3 substituents selected from —O—C₁₋₆alkyl and -Het₆;
   and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from H, and —C₁₋₆alkyl;
A is selected from —(CH₂)ₙ—Y—(CH₂)ₘ—, and —NR₆—, —(C═O)—NR₅—;
X₁ is selected from —O—C₁₋₆alkyl-, —C₁₋₆alkyl-NR₃-, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is —NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4

In a further embodiment, the present invention provides a compound of Formula I or a stereoisomer, tautomer, racemic, metabolite, pro- or predrug, salt, hydrate, N-oxide form, or solvate thereof, for use in the diagnosis, prevention and/or treatment of a RIP2-kinase associated disease wherein
A₁ is N and A₂ is C;
R₁ and R₄₁ are each independently selected from —H, -halo, —C₁₋₆alkyl, —(C═O)—R₄, and —CN;
   wherein each of said —C₁₋₆alkyl is optionally and independently substituted with from 1 to 3 substituents selected from —O—C₁₋₆alkyl;
R₂ is selected from —H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;
R₆ is selected from —C₁₋₆alkyl, —(C═O)—C₁₋₆alkyl, —(C═O)—C₃₋₆cycloalkyl, -Het₆, and —C₃₋₆cycloalkyl;
   wherein each of said —C₁₋₆alkyl is optionally and independently substituted with from 1 to 3 substituents selected from —O—C₁₋₆alkyl and -Het₆;
   and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from —H, and —C₁₋₆alkyl;
A is selected from —(CH₂)ₙ—Y—(CH₂)ₘ—, —NR₆—, and —(C═O)—NR_{S}—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is —NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4

In particular in the compounds according to this invention, the pyrazolopyrimidine or the imidazopyridazine moiety is linked to the aryl or heteroaryl moiety at position Z₄ or Z₅, in accordance with the numbering as provided in Formula I. Furthermore, the R₁ of the compounds according to this invention is preferably linked to the aryl or heteroaryl moiety at position Z₁, Z₂ or Z₃, in accordance with the numbering as provided in Formula I.

In yet a further aspect, the present invention provides a compound selected from the list comprising:

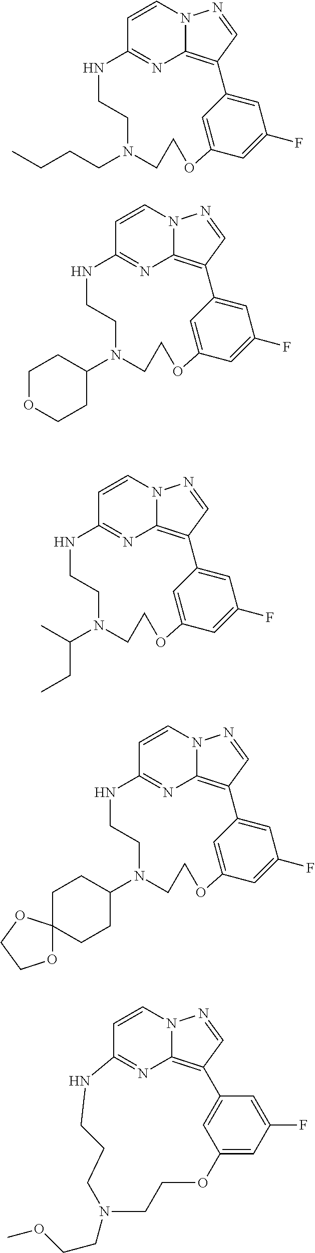

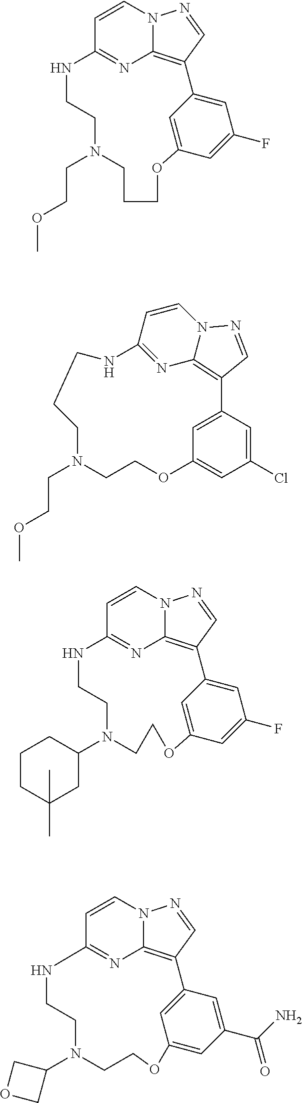

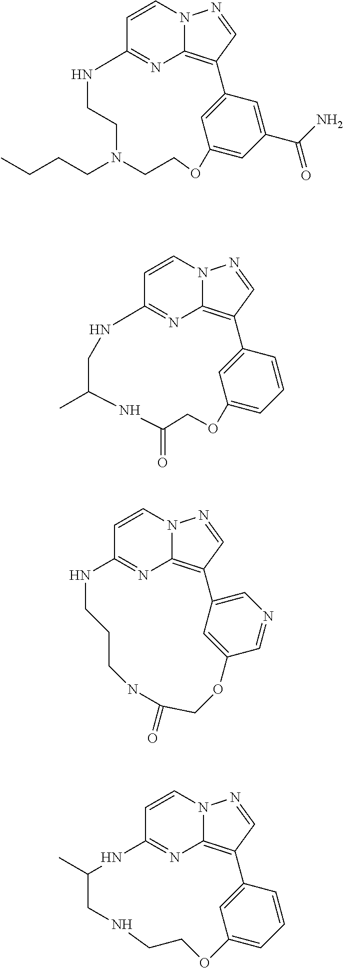 [SMILES: CCCCN1CCNc2ccn3ncc(c3n2)-c2cc(cc(C(N)=O)c2)OCC1 ; CC1CNc2ccn3ncc(c3n2)-c2cccc(c2)OCC(=O)N1 ; O=C1COc2cncc(c2)-c2cnn3ccc(nc23)NCCCN1 ; CC1CNCCOc2cccc(c2)-c2cnn3ccc(nc23)N1]

The compounds of the present invention can be prepared according to the reaction schemes provided in the examples hereinafter, but those skilled in the art will appreciate that these are only illustrative for the invention and that the compounds of this invention can be prepared by any of several standard synthetic processes commonly used by those skilled in the art of organic chemistry.

### Method of Treatment

Compounds of formula (I) a stereoisomer, tautomer, racemic, metabolite, pro- or predrug, salt, hydrate, N-oxide form, or solvate thereof, are inhibitors of RIP2 kinase activity and are thus believed to be of potential use in the diagnosis, prevention and/or treatment of inflammatory disorders, in particular Crohn's disease, bowel disease, Sarcoidosis, psoriasis, rheumatoid arthritis, asthma, ulcerative colitis, lupus, uveitis, blau syndrome, granulomatous inflammation, in particular behget's disease, multiple sclerosis and insulin-resistant type 2 diabetes.

As used herein, the terms “inflammatory disorder” or “inflammatory disease” can refer to a disorder or disease characterized by aberrant activation of the immune system that leads to or causes pathogenesis of several acute and chronic conditions including, for example, sarcoidosis, rheumatoid arthritis, inflammatory bowel disease, transplant rejection, colitis, gastritis and ileitis. An inflammatory disease can include a state in which there is a response to tissue damage, cell injury, an antigen, an infectious disease, and/or some unknown cause. Symptoms of inflammation may include, but are not limited to, cell infiltration and tissue swelling.

In the invention, particular preference is given to compounds of Formula I or any subgroup thereof that in the inhibition assay for RIP2 described below inhibit kinase activity with an IC₅₀ value of less than 10 μM, preferably less than 1 μM, most preferably less than 100 nM.

Said inhibition may be effected in vitro and/or in vivo, and when effected in vivo, is preferably effected in a selective manner, as defined above.

The term “RIP2 kinase-mediated condition” or “disease”, as used herein, means any disease or other deleterious condition in which the RIP2 kinase and/or mutants thereof is/are known to play a role. The term “RIP2 kinase-mediated condition” or “disease” also means those diseases or conditions that are alleviated by treatment with a RIP2 kinase inhibitor. Accordingly, another embodiment of the present invention relates to treating or lessening the severity of one or more diseases in which the RIP2 kinase is known to play a role.

For pharmaceutical use, the compounds of the invention may be used as a free acid or base, and/or in the form of a pharmaceutically acceptable acid-addition and/or base-addition salt (e.g.

obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by a compound of this invention with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

The pharmaceutically acceptable salts of the compounds according to the invention, i.e. in the form of water-, oil-soluble, or dispersible products, include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalene-sulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. In addition, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethylbromides and others. Other pharmaceutically acceptable salts include the sulfate salt ethanolate and sulfate salts.

Generally, for pharmaceutical use, the compounds of the inventions may be formulated as a pharmaceutical preparation or pharmaceutical composition comprising at least one compound of the invention and at least one pharmaceutically acceptable carrier, diluent or excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for administration by inhalation, by a skin patch, by an implant, by a suppository, etc.. Such suitable administration forms which may be solid, semi-solid or liquid, depending on the manner of administration as well as methods and carriers, diluents and excipients for use in the preparation thereof, will be clear to the skilled person; reference is again made to for instance U.S. Pat. No. 6,372,778, U.S. Pat. No. 6,369,086, U.S. Pat. No. 6,369,087 and U.S. Pat. No. 6,372,733, as well as to the standard handbooks, such as the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, creams, lotions, soft and hard gelatin capsules, suppositories, eye drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc.. The compositions may also be formulated so as to provide rapid, sustained or delayed release of the active compound(s) contained therein, for example using liposomes or hydrophilic polymeric matrices based on natural gels or synthetic polymers. In order to enhance the solubility and/or the stability of the compounds of a pharmaceutical composition according to the invention, it can be advantageous to employ α-, β- or γ-cyclodextrins or their derivatives. An interesting way of formulating the compounds in combination with a cyclodextrin or a derivative thereof has been described in EP-A-721,331. In particular, the present invention encompasses a pharmaceutical composition comprising an effective amount of a compound according to the invention with a pharmaceutically acceptable cyclodextrin.

In addition, co-solvents such as alcohols may improve the solubility and/or the stability of the compounds. In the preparation of aqueous compositions, addition of salts of the compounds of the invention can be more suitable due to their increased water solubility.

For local administration, the compounds may advantageously be used in the form of a spray, ointment or transdermal patch or another suitable form for topical, transdermal and/or intradermal administration.

More in particular, the compositions may be formulated in a pharmaceutical formulation comprising a therapeutically effective amount of particles consisting of a solid dispersion of the compounds of the invention and one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

It may further be convenient to formulate the compounds in the form of nanoparticles which have a surface modifier adsorbed on the surface thereof in an amount sufficient to maintain an effective average particle size of less than 1000 nm. Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the compounds according to the invention involves a pharmaceutical composition whereby the compounds are incorporated in hydrophilic polymers and applying this mixture as a coat film over many small beads, thus yielding a composition with good bio-availability which can conveniently be manufactured and which is suitable for preparing pharmaceutical dosage forms for oral administration. Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The preparations may be prepared in a manner known per se, which usually involves mixing at least one compound according to the invention with the one or more pharmaceutically acceptable carriers, and, if desired, in combination with other pharmaceutical active compounds, when necessary under aseptic conditions. Reference is again made to U.S. Pat. No. 6,372,778, U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the invention, e.g. about 10, 25, 50, 100, 200, 300 or 400 mg per unit dosage.

The compounds can be administered by a variety of routes including the oral, rectal, ocular, transdermal, subcutaneous, intravenous, intramuscular or intranasal routes, depending mainly on the specific preparation used and the condition to be treated or prevented, and with oral and intravenous administration usually being preferred. The at least one compound of the invention will generally be administered in an “effective amount”, by which is meant any amount of a compound of Formula or any subgroup thereof that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight day of the patient per day, more often between 0.1 and 500 mg, such as between 1 and 250 mg, for example about 5, 10, 20, 50, 100, 150, 200 or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778,U.S. Pat. No. 6,369,086, U.S. Pat. No. 6,369,087 and U.S. Pat. No. 6,372,733 and the further prior art mentioned above, as well as to the standard handbooks, such as the latest edition of Remington's Pharmaceutical Sciences.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules.

Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous administration, the compound according to the invention, if desired with the substances customary therefore such as solubilizers, emulsifiers or further auxiliaries are brought into solution, suspension, or emulsion. The compounds of the invention can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquefy and/or dissolve in the rectal cavity to release the drug.

In preferred embodiments, the compounds and compositions of the invention are used orally or parenterally.

The invention will now be illustrated by means of the following synthetic and biological examples, which do not limit the scope of the invention in any way.

EXAMPLES

A. Compound Synthesis and Physicochemical Properties

The compounds of this invention can be prepared by any of several standard synthetic processes commonly used by those skilled in the art of organic chemistry. The compounds are generally prepared from starting materials which are either commercially available or prepared by standard means obvious to those skilled in the art.

General Schemes:

As indicated herein before, the present invention provides compounds according to formula I, for use in the diagnosis, prevention and/or treatment of RIP2-kinase associated diseases:

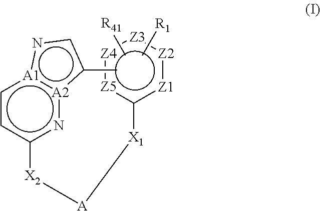

With reference to the general reaction schemes suitable for preparing said compounds, these compounds can be represented by formulas la or lb respectively, for which the general reaction schemes can be found herein below.

In general the compounds of formula (I) can be prepared as shown in scheme 1 below wherein a pyrazolo[1,5-a]pyrimidine or a imidazo[2,1-f]pyridazine of formula (II) is converted by reaction with a compound of formula (III) into a compound of formula (IV), which is then reacted with a (hetero-) aryl of formula (V) to form a compound of formula (VI). The compound of formula (VI) can then be optionally deprotected if desired before cyclisation to form a compound of formula (VII). The compound of formula (VII) can be optionally converted into a compound of general formula (I).

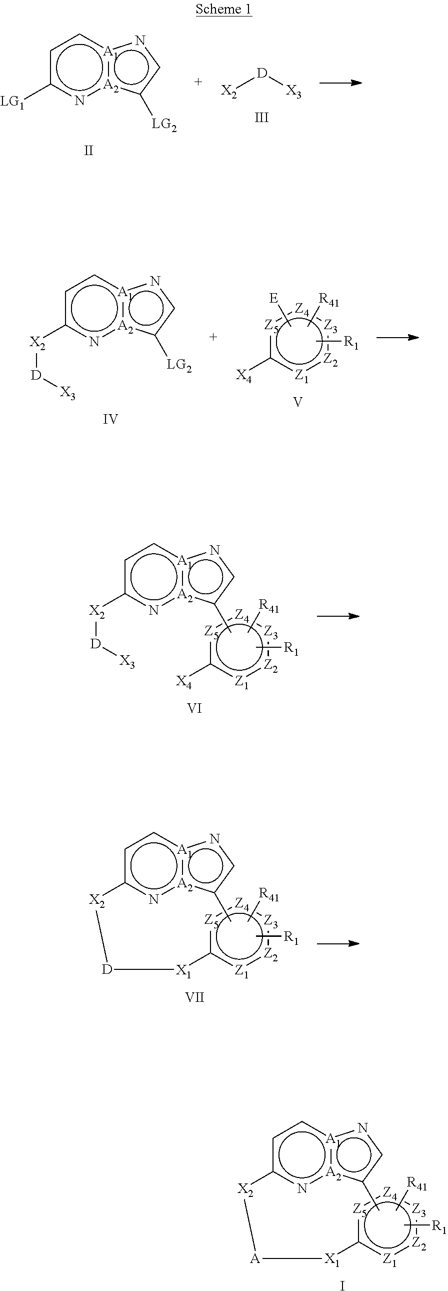

In the above scheme:

LG₁ and LG₂ each independently represent suitable leaving or functional groups;

X₃ and X₄ together with the functional moiety to which they are attached represent an unprotected or a protected functional group which upon reaction (after deprotection) produce together X₁ as defined in formula I;

E represents a suitable functional group that can be used to form a direct bond between the (hetero-)aryl group and the scaffold.

D represents a functional group such as A or a protected functional group, which upon further reaction and/or deprotection produces a functional group such as A as defined in formula I;

In the above reaction of the compound of formula (II) with the compound of formula (III) the leaving groups LG₁ and LG₂ are advantageously a halo group such as a chlorine or a bromine group. The reaction can be affected by a substitution for example by treating the compound of formula (II) with the compound of formula (III) in an organic solvent such as acetonitrile with an appropriate base such as for example diisopropylethylamine at an elevated temperature for example under reflux.

Compounds of formula (III) can be obtained through various selective protection and deprotection steps.

The compound of formula (IV) can optionally be protected with a suitable protecting group such as a tert-butyloxycarbonylamino group in a conventional manner for example by treatment with tert-butoxycarbonyl anhydride in basic conditions using for example triethylamine and 4-(dimethylamino)pyridine in a solvent such as tetrahydrofurane at an elevated temperature such as under reflux.

The reaction of the resulting compound (IV) with a (hetero-)aryl compound of formula (V) is advantageously effected through the coupling of a boronic acid E or boronic ester E derivative of the (hetero-)aryl compound under Suzuki conditions using for example tetrakis(triphenylphosphine)pallad ium(0), 2-dicyclohexylphosph ino-2′,4′,6′-triisopropylbiphenyl

(Xphos) and potassium phosphate tribasic in a solvent mixture such as 1,4-dioxane/water at an elevated temperature for example under reflux.

The resulting compound of formula (VI) can optionally be treated to remove any desired protecting groups for example silyl ether groups such as tert-butyldimethylsilyl groups can be converted to the parent free hydroxy group. Such deprotection can be effected in a conventional manner for example using tetrabutylammonium fluoride in tetrahydrofuran at room temperature. The resulting compound of formula (VI) can also optionally be treated to remove any desired protecting groups for example benzyl groups can be removed in a conventional manner for example using hydrogen gas and palladium on activated charcoal (10%) in a solvent such as methanol at a temperature such as room temperature. The compound of formula (VI) can optionally be treated to remove any desired protecting groups for example tert-butyloxycarbonylamino groups can be converted to the parent free amino group. Such deprotection can be effected in a conventional manner for example by treatment under acidic conditions for example using a 4N acetyl chloride solution in a solvent such as methanol at for example room temperature.

The cyclisation of the compound of formula (VI) can be effected for example under Mitsunobu conditions using for example diisopropyl azodicarboxylate and triphenylphosphine in a solvent mixture such as 2-methyl-1,4-dioxane and toluene at an elevated temperature such as 90° C. The resulting compound of formula (VII) can optionally be treated to remove any desired protecting groups for example tert-butyloxycarbonylamino groups can be converted to the parent free amino group. Such deprotection can be effected in a conventional manner for example by treatment under acidic conditions for example using a 4N hydrochloric acid solution in methanol at room temperature.

The deprotected compound can optionally be treated to form an amide compound of formula (I). The reaction can advantageously be affected by treatment with an acylchloride and a base such as triethylamine in a solvent such as tetrahydrofuran at room temperature. The reaction can also be affected using for example O-(benzotriazol-1-yl)-N,N,N′,N′-tetramethyluronium hexafluorophosphate (HBTU) and diisopropylethylamine in a solvent such as N,N-dimethylformamide at for example room temperature.

Compounds B19, B21, B76, F81, F82, F83, F84, F86, F87, F88, F89, F91 and F92 may be prepared according to the synthesis described in Scheme 1.

The compounds of formula (I) can also be prepared as shown in general scheme 2 below wherein a pyrazolo[1,5-a]pyrimidine or a imidazo[2,1-f]pyridazine of formula (II) is converted by reaction with a compound of formula (VIII) into a compound of formula (IX). The compound of formula (IX) can be optionally be converted into a compound of formula (IV) which is then reacted with a (hetero-)aryl of formula (V) to form a compound of formula (VI). The compound of formula (VI) can then be optionally deprotected if desired before cyclisation to form a compound of formula (VII). The compound of formula (VII) can be optionally converted into a compound of general formula (I).

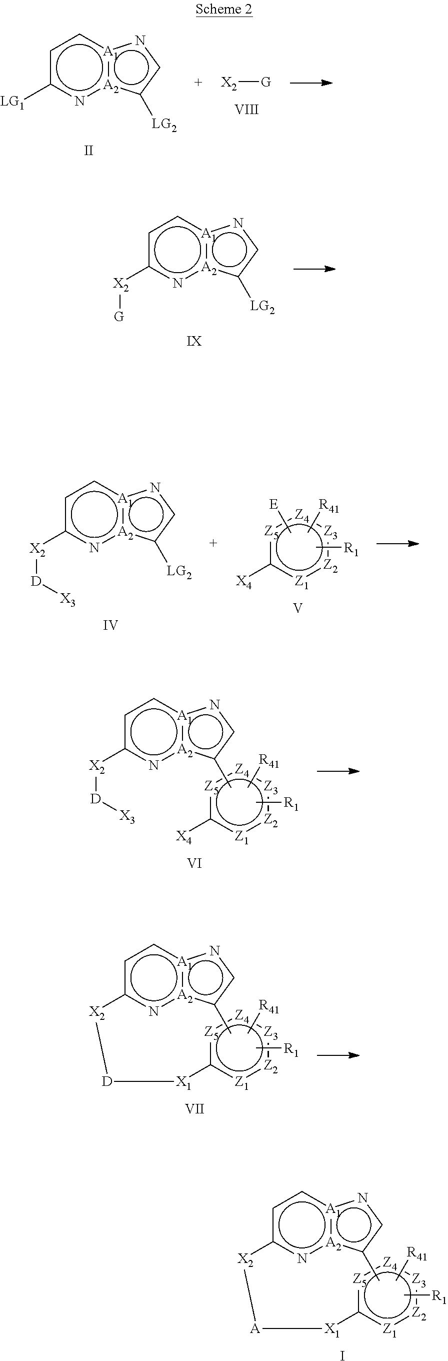

In the above scheme:

LG₁ and LG₂ each independently represent suitable leaving or functional groups;

E represents a suitable functional group that can be used to form a direct bond between the (hetero-)aryl group and the scaffold.

G represents a suitable functional group or protected functional group, which upon further reaction and/or deprotection produces a functional group such as D;

D represents a functional group such as A or a protected functional group, which upon further reaction and/or deprotection produces a functional group such as A as defined in formula I;

In the above reaction of the compound of formula (II) with the compound of formula (VIII) the leaving groups LG₁ and LG₂ are advantageously a halo group such as a chlorine or a bromine group. The reaction can be affected by a substitution for example by treating the compound of formula (II) with the compound of formula (VIII) in an organic solvent such as tetrahydrofuran with an appropriate base such as for example sodium hydride at for example room temperature.

Compounds of formula (VIII) can be either commercially acquired or obtained through various selective protection and deprotection steps.

The compounds of formula (IX) can be deprotected using for example acidic conditions such as a 4N hydrochloric acid solution in methanol at room temperature.

The compounds of formula (IX) can be converted into compounds of formula (IV) by using for example a reductive amination. The reaction can be affected by treating the compound of formula (IX) with an alhyde in the presence of a reducing agent such as sodium triacetoxy borohydride and a base such as triethylamine in a solvent such as dichloromethane at for example room temperature.

The reaction of the compound with formula (IV) with a (hetero-)aryl compound of formula (V) is advantageously effected under Suzuki conditions using for example tetrakis(triphenylphosphine)palladium(0) and potassium phosphate tribasic in a solvent mixture such as 1,4-dioxane/water at an elevated temperature for example 80° C.

The resulting compound of formula (VI) can optionally be treated to remove any desired protecting groups for example silyl ether groups such as tert-butyldimethylsilyl groups can be converted to the parent free hydroxy group. Such deprotection can be effected using for example acetic acid in tetrahydrofuran at for example room temperature. The compound of formula (VI) can optionally be treated to remove any desired protecting groups for example tert-butyloxycarbonylamino groups can be converted to the parent free amino group. Such deprotection can be effected in a conventional manner for example by treatment under acidic conditions for example using a 4N acetyl chloride solution in a solvent such as methanol at for example room temperature.

The free hydroxyl group can be converted into a leaving group such as a chloride by reacting the hydroxyl group for example with thionyl chloride in the presence of a base such as pyridine in a solvent such as dichloromethane at an elevated temperature for example under reflux.

The cyclisation of the compound of formula (VII) can be advantageously effected under Williamson conditions using a base such as cesium carbonate in a solvent such as N,N-dimethylformamide at an elevated temperature such as 90° C. Other condtions that can be used for the cyclisation of the compound of formula (VII) can be for example by treatment with O-(benzotriazol-1-yl)-N,N,N′,N′-tetramethyluronium hexafluorophosphate (HBTU) and N,N-diisopropylethylamine in a solvent such as N,N-dimethylformamide at for example room temperature.

The resulting compound of formula (VII) can optionally be treated to form a compound of formula (I).

Compound B74 may be prepared according to the synthesis described in Scheme 2.

The compounds of formula (I) can also be prepared as shown in general scheme 3 below wherein a pyrazolo[1,5-a]pyrimidine or a imidazo[2,1-f]pyridazine of formula (II) is converted by reaction with a compound of formula (VIII) into a compound of formula (IX). The compound of formula (IX) can be optionally reacted with a (hetero-)aryl of formula (V) to form a compound of formula (X). The compound of formula (X) can be converted into the compounds of formula (XI). The compound of formula (XI) can then be optionally deprotected if desired before cyclisation to form a compound of formula (VII). The compound of formula (VII) can be optionally converted into a compound of general formula (I).

In the below scheme 3:

LG₁ and LG₂ each independently represent suitable leaving or functional groups;

X₄ and X₅ together with the functional moiety to which they are attached represent an unprotected or a protected functional group which upon reaction (after deprotection) produce together X₁ as defined in formula I;

E represents a suitable functional group that can be used to form a direct bond between the (hetero-)aryl group and the scaffold.

G and J represent functional groups or protected functional groups, which upon further reaction and/or deprotection produce a functional group such as D;

D represents a functional group such as A or a protected functional group, which upon further reaction and/or deprotection produces a functional group such as A as defined in formula I;

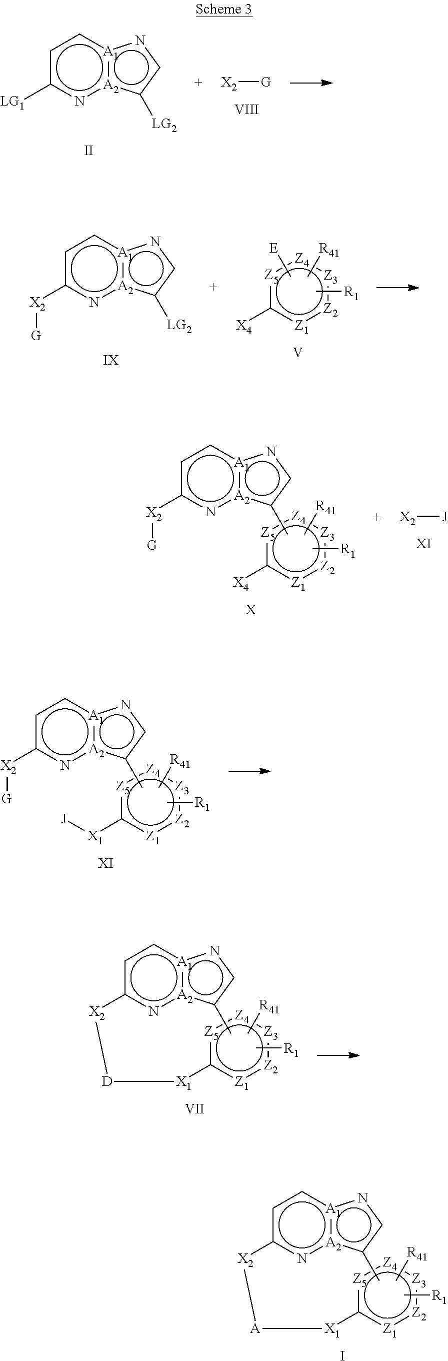

In the above reaction of the compound of formula (II) with the compound of formula (VIII) the leaving groups LG, and LG₂ are advantageously a halo group such as a chlorine or a bromine group. The reaction can be affected by a substitution for example by treating the compound of formula (II) with the compound of formula (VIII) in an organic solvent such as acetonitrile with an appropriate base such as for example diisopropylethylamine at an elevated temperature for example under reflux.

Compounds of formula (VIII) and (XI) can be either commercially acquired or obtained through various selective protection and deprotection steps.

The resulting compound of formula (IX) can optionally be protected with a suitable protecting group such as a tert-butyloxycarbonylamino group in a conventional manner for example by treatment with tert-butoxycarbonyl anhydride in basic conditions using for example triethylamine and 4-(dimethylamino)pyridine in a solvent such as tetrahydrofuran at an elevated temperature such as under reflux.

The reaction of the resulting compound (IX) with a (hetero-)aryl compound of formula (V) is advantageously effected through the coupling of a boronic acid E or boronic ester E derivative of the (hetero-)aryl compound under Suzuki conditions using for example tetrakis(triphenylphosphine)palladium(0), 2-dicyclohexylphosph ino-2′,4′,6′-triisopropylbiphenyl (Xphos) and potassium phosphate tribasic in a solvent mixture such as 1,4-dioxane/water at an elevated temperature for example 80° C.

The reaction of the resulting compound of formula (X) with a compound of formula (XI) which can be advantageously effected under Williamson conditions using a base such as potassium carbonate in a solvent such as acetonitrile at an elevated temperature such as under reflux. This reaction can also be effected under Mitsunobu conditions using for example diisopropyl azodicarboxylate and triphenylphosphine in a solvent such as tetrahydrofuran at an elevated temperature such as 90° C.

The resulting compound of formula (XI) can optionally be treated to remove any desired protecting groups for example tert-butyloxycarbonylamino groups can be converted to the parent free amino group and for example ester groups can be converted to the parent free carboxylic acid groups. Such deprotection can be effected in a conventional manner for example by treatment under acidic conditions for example using an aqueous 6N hydrochloric acid solution in a solvent such as acetoniitrile at an elevated temperature for example 60° C. or using an acid such as trifluoroacetic acid in a solvent such as dichloromethane at for example room temperature.

The cyclisation of the compound of formula (XI) can be effected for example by treatment with O-(benzotriazol-1-yl)-N,N,N′,N′-tetramethyluronium hexafluorophosphate (HBTU) and N,N-diisopropylethylamine in a solvent such as N,N-dimethylformamide at for example room temperature.

The resulting compound of formula (VII) can optionally be treated to form a compound of formula (I).

Compounds B36, B48, F105, F106 and F108 may be prepared according to the synthesis described in Scheme 3.

The above general processes are illustrated by the specific processes which are described in the patent applications WO2013/045653 A1 and WO2013/046029 A1.

Preparation of Intermediate F78

Intermediate F78 is prepared following general scheme 1.

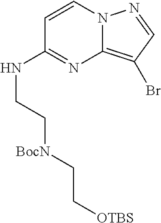 [SMILES: CC(C)(C)OC(=O)N(CCNc1ccn2ncc(Br)c2n1)CCO[Si](C)(C)C(C)(C)C]

Step A

To a solution of 3-bromo-5-chloropyrazolo[1,5-a]pyrimidine (14.0 g, 60.22 mmol, leg), the linker (synthesis described in the patent WO2013/045653 A1; preparation of intermediate 21) (21.1 g, 66.24 mmol, 1.1 eq) and DIPEA (13.67 ml, 78.29 mmol, 1.3 eq) in acetonitrile (180 ml) was heated at 70/80 ° C. for 18 h. Upon completion, monitored by TLC plate, the reaction mixture was concentrated. The residue was dissolved in EtOAc and washed 2× with water and once with Brine. The organic layer was dried (MgSO4), filtered, concentrated. The crude product was further purified by flash chromatography using a eluent a gradient: Heptane:EtOAc 100:0 to 80:20 fast to 60:40 slow. The product fractions were collected and concentrated to obtain 23.6 g of a brown solid (76% yield).

MH+: 514.2/516.2

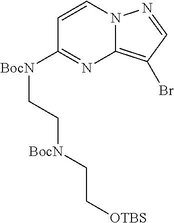 [SMILES: CC(C)(C)OC(=O)N(CCO[Si](C)(C)C(C)(C)C)CCN(C(=O)OC(C)(C)C)c1ccn2ncc(Br)c2n1]

Step B

The title compound from step A, Boc anhydride (15.01 g, 68.8 mmol, 1.5 eq) and DMAP (0.28 g, 2.29 mmol, 0.05 eq) were dissolved in THF (137 ml) and the mixture was heated at 65° C. for 4 h. Upon completion, monitored by TLC plate, the reaction mixture was concentrated. The crude product was further purified by flash chromatography using as eluent a gradient: Heptane:EtOAc 100:0 to 50:50 fast 6c volumes. The product fractions were collected and concentrated to obtain 27.0 g of brown oil (96% yield).

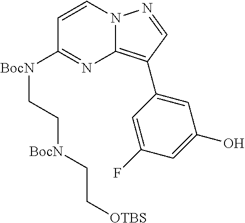 [SMILES: CC(C)(C)OC(=O)N(CCO[Si](C)(C)C(C)(C)C)CCN(C(=O)OC(C)(C)C)c1ccn2ncc(-c3cc(O)cc(F)c3)c2n1]

Step C

A mixture of the title compound from step B, (3-fluoro-5-hydroxyphenyl)boronic acid (1.78 g, 11.39 mmol, 1.0 eq), XPhos (0.32 g, 0.68 mmol, 0.06 eq) and potassium phosphate (7.2 g, 33.92 mmol, 3.0 eq) were dissolved in Dioxane /water 3:1 and degassed with N2. Then Palladium Tetrakis (0.39 g, 0.34 mmol, 0.03 eq) was added to the stirring solution. The resulting reaction mixture was stirred at 80° C. for 6 h under N2 atmosphere. To reach completion, additional amounts of the Boronic acid (1.0 eq), Palladium Tetrakis (0.03 eq) and XPhos (0.06 eq) were added. The reaction mixture was stirred for an additional 18 h at 90° C.

The mixture was diluted with EtOAc and the layers were separated. The organic layer was washed 2× with water and once with Brine, dried (MgSO4), filtered, concentrated. The crude product was further purified by flash chromatography using as eluent a gradient: Heptane:EtAOc. 100:0 to 60:40. The product fractions were collected and concentrated to obtain 7.2 g of a solid (98% yield). MH+:546.3

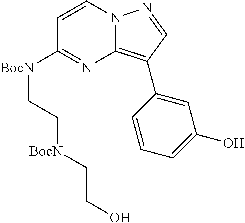 [SMILES: CC(C)(C)OC(=O)N(CCO)CCN(C(=O)OC(C)(C)C)c1ccn2ncc(-c3cccc(O)c3)c2n1]

Step D

To a solution of the title compound from Step C in THF (33 ml) was added a solution of TBAF 1M in THF (14.5 ml, 14.5 mmol). The reaction mixture was stirred for 18 h at RT, and then the solvent was concentrated to dryness. The residue was dissolved in Ethyl acetate and washed 3times with water and once with brine. The organic layer was dried over Magnesium sulfate, filtered, concentrated. The crude product was further purified by flash chromatography (n-Hp:EA 0:20 to 30:70) to obtain the title compound as a white solid (5.0 g, 84% yield).

MH+:432.2

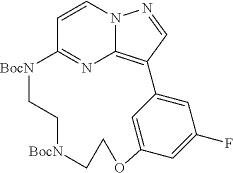 [SMILES: CC(C)(C)OC(=O)N1CCOc2cc(F)cc(c2)-c2cnn3ccc(nc23)N(C(=O)OC(C)(C)C)CC1]

Step E

To a stirring solution of Triphenylphosphine (7.66 g, 29.22 mmol) in Toluene (44 ml) at 90° C. were simultaneously added a solution of the title compound from Step D (5.0 g, 9.74 mmol) in 2-MeTHF (11.6 ml) and a solution of DIAD (5.79 ml, 29.22 mmol) in Toluene (11.6 ml) over 5 h. The resulting mixture was further stirred at 90° C. for 30 minutes. The reaction mixture was concentrated to dryness and directly engaged in the next step without purification.

MH+: 514.3

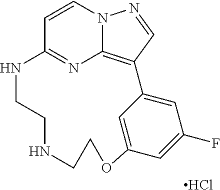 [SMILES: Fc1cc2cc(c1)-c1cnn3ccc(nc13)NCCNCCO2 ; Cl]

Step F

To the title compound from step E (9.8 g, 19.08 mmol) was added a 4M solution of HCl in MeOH (57 ml). The resulting mixture was stirred at RT for 18 h then at 40° C. for 8 h. At RT the white slurry was filtered off and washed with diisopropylether. The solid was dried under vacuum to obtain the title compound as a white solid (3.0 g, 88% yield over 2 steps).

Melting point: >300° C., decomposition

MH+: 314.10

Preparation of Intermediate F79

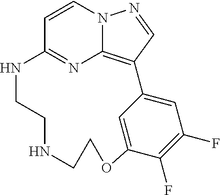 [SMILES: Fc1cc2cc(c1F)OCCNCCNc1ccn3ncc-2c3n1]

is prepared following general scheme 1 and according to the procedures described in the patent application WO2013/045653 A1 to obtain example 17.

Preparation of Intermediate F80

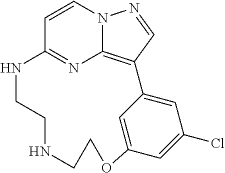 [SMILES: Clc1cc2cc(c1)-c1cnn3ccc(nc13)NCCNCCO2]

is prepared following general scheme 1 and according to the procedures described in the patent application WO2013/045653 A1 to obtain example 17.

Examples F81 to F89 were prepared following general scheme 1 and according to the procedures described in the patent application WO2013/045653 A1 to obtain example 6.

Preparation of Intermediate F90

Intermediate F90 is Prepared Following General Scheme 1

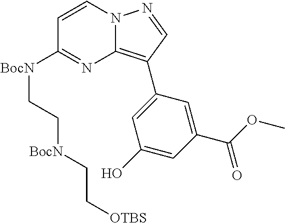 [SMILES: COC(=O)c1cc(O)cc(-c2cnn3ccc(N(CCN(CCO[Si](C)(C)C(C)(C)C)C(=O)OC(C)(C)C)C(=O)OC(C)(C)C)nc23)c1]

Step A

A mixture of Dioxane and water (3:1) (148 ml) was placed in a flask and degased by bubbling nitrogen gas. Then the title compound from Example F78 step B (15 g, 24.4 mmol, 1.0 eq), the Boronic ester (8.82 g, 31.73 mmol, 1.3 eq), Palladium Tetrakis (0.568 g, 0.49 mmol, 0.02 eq), XPhos (0.93 g, 1.95 mmol, 0.08 eq) and Potassium phosphate (25.9 g, 5.0 eq) were added and the suspension was stirred at 85° C. under nitrogen for 15 hours. Upon completion, monitored by LCMS, Dioxane was removed, water was added and the product was extracted with ethyl acetate. The organic layer was dried over magnesium sulfate, filtered and the filtrate was concentrated under reduce pressure. The product was purified by flash chromatography on silica gel using as eluents mixtures of heptane: ethyl acetate (from 0% to 33% of ethyl acetate). The product fractions were collected and the solvent was evaporated to dryness. The title compound was obtained as a solid (13.43 g, 80.2% yield).

MH+: 586.1

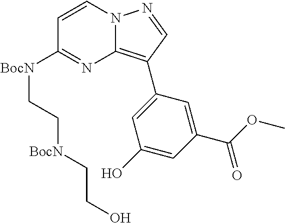 [SMILES: COC(=O)c1cc(O)cc(-c2cnn3ccc(N(CCN(CCO)C(=O)OC(C)(C)C)C(=O)OC(C)(C)C)nc23)c1]

Step B

A solution of the title compound from step C and 1M TBAF (21.54 ml, 1 eq) in THF (59 ml) was stirred 10 at r.t. for 1 hour. Upon completion, monitored by LCMS, solvent was removed under reduced pressure and the residue was dissolved in ethyl acetate, washed with water (×3) and brine. Organic layer was washed with brine, dried over magnesium sulfate and evaporated to dryness. The product was used as such in the next reaction step.

MH+: 572.0

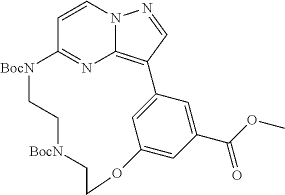 [SMILES: COC(=O)c1cc2cc(c1)-c1cnn3ccc(nc13)N(C(=O)OC(C)(C)C)CCN(C(=O)OC(C)(C)C)CCO2]

Step C

The reaction was performed in parallel in 2 batches.

A solution of the title compound from step D (8.95 g, 15.65 mmol) in 2-methyl THF (20 ml/mmol) and a solution of DIAD (9.31 ml, 46.95 mmol, 3.0 eq) in toluene (same volume) were simultaneously added 20 to a solution of Triphenylphosphine (12.31 g, 46.95 mmol, 3.0 eq) in toluene (75 ml/mmol of starting material A) at 90° C. for 3 hours. After that, the reaction mixture was heated for 30 minutes. Upon completion, monitored by LCMS, solvent was evaporated and the product was purified by flash chromatography on silica gel using as eluents mixtures of dichloromethane: methanol (from 0% to 10% of methanol). The product fractions were collected and the solvent was evaporated to dryness 25 to lead the expected compound in 7.7 g, 88% yield.

MH+: 554.0

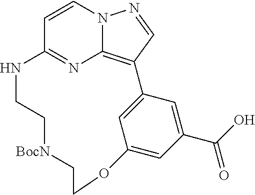 [SMILES: CC(C)(C)OC(=O)N1CCNc2ccn3ncc(c3n2)-c2cc(cc(C(=O)O)c2)OCC1]

Step D

A mixture of the title compound from step E (1.5 g, 2.71 mmol, 1.0 eq) and lithium hydroxide hydrate (0.34 g, 8.13 mmol, 3.0 eq) were suspended in THF/MeOH/H2O (2:2:1) (25 ml). The mixture was stirred at 50 ° C. for 15 hours. Upon completion, monitored by LCMS, solvent was removed. Water was added and HCl 1M was added to acidify the solution to pH 6. The resulting solid was filtered and washed with methanol, then dried at high vacuum (615 mg).

The product in the aqueous phase was extracted with dichloromethane. The organic layer was dried over magnesium sulfate, filtered and the filtrate was concentrated under reduce pressure. The product was purified by flash chromatography on silica gel using as eluents mixtures of DCM:MeOH (from 0% to 100% of methanol) and then dichloromethane: methanol (from 0 to 10% of methanol). The title compound was obtained as a white solid (917 mg, 77% yield) MH+: 440.0

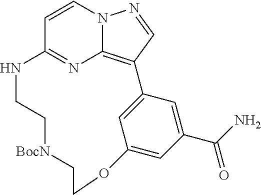 [SMILES: CC(C)(C)OC(=O)N1CCNc2ccn3ncc(c3n2)-c2cc(cc(C(N)=O)c2)OCC1]

Step E

HBTU (0.637 g, 1.68 mmol, 1.2 eq) was added to a solution of the title compound from step F (0.615 mg, 1.40 mmol, 1.0 eq), Ammonium chloride (0.08 g, 1.40 mmol, 1.10 eq) and DIPEA (0.595 ml, 3.50 mmol, 2.5 eq) in DMF (4 ml). The mixture was stirred at RT for 19 hours. Upon completion, monitored by LCMS, the reaction was diluted with ethyl acetate and washed with NaHCO3 saturated solution. The organic layer was dried over magnesium sulfate, filtered and the filtrate was concentrated under reduce pressure. The product was purified by flash chromatography on silica gel using as eluents mixtures of heptane:ethyl acetate (from 0% to 100% of ethyl acetate). The product fractions were collected and the solvent was evaporated to dryness to lead the title compound as a solid (507 mg, 82%).

MH+: 439.0

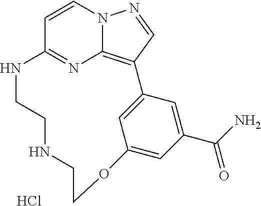 [SMILES: NC(=O)c1cc2cc(c1)-c1cnn3ccc(nc13)NCCNCCO2 ; Cl]

Step F

The title compound from step G (507 mg, 1.16 mmol, 1.0 eq) was stirred in 4M HCl in Dioxane (3.5 ml) at room temperature for 3 hours. Upon completion, monitored by LCMS, solvent was removed. Ethyl ether was added and the solid formed was filtered of and dried under vacuum to lead the title compound as white solid (372 mg, 85%).

MH+: 339.0

HPLC retention time: 0.197 min

Melting point:

Examples F91 to F92 were prepared following general scheme 1 and according to the procedures described in the patent application WO2013/045653 A1 to obtain example 6.

Preparation of Intermediate F104

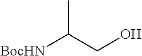

Step A

Boc anhydride (15.98 g, 73.23 mmol, 1.1 eq) was added to a solution of 2-aminopropan-1-ol (5.0 g, 66.57 mmol, 1.0 eq) in CH2Cl2 (200 ml). The mixture was stirred at room temperature for 1 hour. Upon completion, monitored by TLC plate, the product was purified by flash chromatography on silica gel using as eluents mixtures of heptane: ethyl acetate (from 0% to 50% of ethyl acetate). The product fractions were collected and the solvent was evaporated to dryness to lead the title compound in 10.89 g (93% yield).

MH+: 198.1 (M+H+Na)

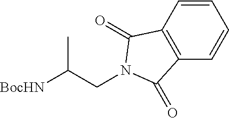 [SMILES: CC(CN1C(=O)c2ccccc2C1=O)NC(=O)OC(C)(C)C]

Step B

The title compound from step A (10.89 g, 62.13 mmo, 1.0 eq) and Pthalimide (13.71 g, 93.2 mmol, 1.5 eq) were dissolved in anhydrous THF (167 ml). The reaction was degassed and Triphenylphosphine (24.44 g, 93.2 mmol, 1.5 eq) was added. The reaction was cooled to 0° C. under N2 atmosphere. DIAD (18.84 g, 93.19 mmol, 1.5 eq) was diluted with 20 ml of THF and added dropwise (exothermic). When the addition was completed, the reaction was allowed to reach room temperature and stirred for 90 minutes. Upon completion, monitored by LCMS, solvent was removed, acetonitrile was added, heated until complete solution and then cooled. The solid thus formed was filtered and dried under vacuum to lead a first fraction in 7.68 g.

The product in the mother liquor was purified by flash chromatography on silica gel using as eluents mixtures of heptane: ethyl acetate (from 0% to 50% of ethyl acetate). The product fractions were collected and the solvent was evaporated to dryness to lead the title compound as a solid in 7.892 g. It contains same impurities related with DIAD.

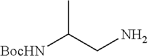

Step C

A solution of the title compound from step B (9.0 g, 29.57 mmol, 1.0 eq) and Hydrazine hydrate (2.76 ml, 88.71 mmol, 3.0 eq) in ethanol (89 ml) was stirred at 70° C. for 4 hours. Upon completion, monitored by LCMS, the reaction mixture was cooled to RT; the resulting suspension was filtered to remove the white solid formed. The filtrate was then evaporated and the residue was dissolved in ethyl acetate, washed with NaOH 1M and brine. Organic layer was dried, filtered and concentrated to give the title compound as colorless oil, which was used as such in the next synthetic step.

### Example F105

Example F105 was Prepared Following General Scheme 3

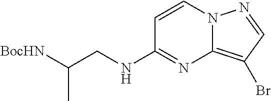 [SMILES: CC(CNc1ccn2ncc(Br)c2n1)NC(=O)OC(C)(C)C]

Step A

A solution of 3-bromo-5-chloropyrazolo[1,5-a]pyrimidine (3.0 g, 12.9 mmol, 1.0 eq), the intermediate F104 (4.49 g, 25.8 mmol, 2.0 eq) and DIPEA (4.61 ml, 27.09 mmol, 2.1 eq) in acetonitrile (39 ml) was refluxed for 15 hours. Upon completion, monitored by LCMS, solvent was removed. Ethyl acetate was added and washed with water. The organic layer was dried over magnesium sulfate, filtered and the filtrate was concentrated under reduce pressure. The product was purified by flash chromatography on silica gel using as eluents mixtures of heptane: ethyl acetate (from 0% to 66% of ethyl acetate). The product fractions were collected and the solvent was evaporated to dryness to lead the title compound as a solid in 4.04 g (84.5% yield).

MH+: 370.1/372.1

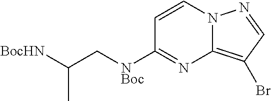 [SMILES: CC(CN(C(=O)OC(C)(C)C)c1ccn2ncc(Br)c2n1)NC(=O)OC(C)(C)C]

Step B

Boc anhydride (2.59 g, 11.86 mmol, 1.1 eq) was added to a mixture of the title compound from step A (3.99 g, 10.78 mmol, 1.0 eq), Triethylamine (1.79 ml, 12.94 mmol, 1.2 eq) and DMAP (66 mg, 0.54 mmol, 0.05 eq) in THF (32 ml). The solution was refluxed for 150 minutes. Upon completion, monitored by LCMS, solvent was removed. Water was added and the product was extracted with ethyl acetate. The organic layer was dried over magnesium sulfate, filtered and the filtrate was concentrated under reduce pressure. The product was purified by flash chromatography on silica gel using as eluents mixtures of heptane: ethyl acetate (from 5% to 40% of ethyl acetate). The product fractions were collected and the solvent was evaporated to dryness to lead the title compound in 4.63 g (91% yield).

MH+: 492.1/494.1

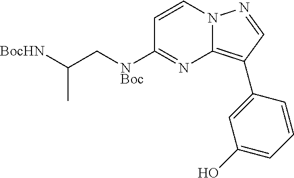 [SMILES: CC(CN(C(=O)OC(C)(C)C)c1ccn2ncc(-c3cccc(O)c3)c2n1)NC(=O)OC(C)(C)C]

Step C

A mixture of Dioxane and Water (3:1) (126 ml) was placed in a flask and degased by bubbling nitrogen gas. Then the title compound from step B (4.63 g, 9.84 mmol, 1.0 eq), 3-hydroxyphenyl Boronic acid (1.76 g, 12.79 mmol, 1.3 eq), Palladium Tetrakis (228 mg, 0.197 mmol, 0.02 eq), XPhos (377 mg, 0.79 mmol, 0.08 eq) and Potassium phosphate (0.223 g, 49.2 mmol, 5.0 eq) were added and the suspension was stirred at 85 ° C. under nitrogen for 3 hours. Upon completion, monitored by LCMS, Dioxane was removed. Water was added and the product was extracted with ethyl acetate. The organic layer was dried over magnesium sulfate, filtered and the filtrate was concentrated under reduce pressure. The product was purified by flash chromatography on silica gel using as eluents mixtures of dichloromethane: methanol (from 100:0 to 20:1). The product fractions were collected and the solvent was evaporated to dryness to lead the title compound in 4.39 g (92% yield). It contains some OPPH3.

MH+: 484.3

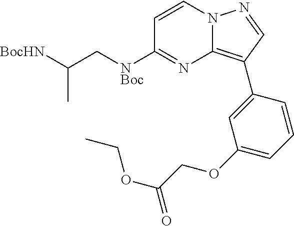 [SMILES: CCOC(=O)COc1cccc(-c2cnn3ccc(N(CC(C)NC(=O)OC(C)(C)C)C(=O)OC(C)(C)C)nc23)c1]

Step D

A mixture of the title compound from step C (1.5 g, 3.1 mmol, 1.0 eq), ethyl 2-bromoacetate (514 ul, 4.65 mmol, 1.5 eq), Potassium carbonate (857 mg, 6.2 mmol, 2.0 eq) and Potassium iodide (27 mg, 0.16 mmol, 0.05 eq) were heated at 80° C. for 2 hours in DMF (9.3 ml). Upon completion, monitored by LCMS, water was added and the product was extracted with ethyl acetate. The organic layer was dried over magnesium sulfate, filtered and the filtrate was concentrated under reduce pressure. The product was purified by flash chromatography on silica gel using as eluents mixtures of heptane: ethyl acetate (from 5% to 33% of ethyl acetate). The product fractions were collected and the solvent was evaporated to dryness to lead the title compound in 1.31 g (74% yield).

MH+: 592.3

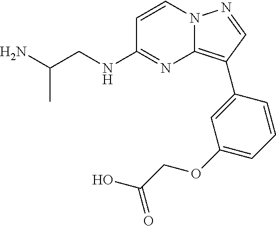 [SMILES: CC(N)CNc1ccn2ncc(-c3cccc(OCC(=O)O)c3)c2n1]

Step E

To a solution of the title compound from step D (1.31 g, 2.29 mmol, 1.0 q) in THF (12 ml/mmol) (6.87 ml) was added HCl 6M (12 ml/mmol). The mixture was stirred at 60 ° C. in a sealed tube for 3 hours. Upon completion, monitored by LCMS, solvent was removed. Toluene/THF (1:1) was added and evaporated. Then toluene was added and evaporated and finally ethanol was added and evaporated. The crude was dried under vacuum and used as such in the next reaction step.

MH+: 342.2

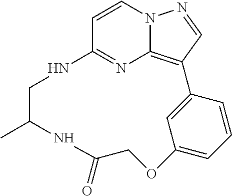 [SMILES: CC1CNc2ccn3ncc(c3n2)-c2cccc(c2)OCC(=O)N1]

Step F

A suspension of the title compound from step E (2.02 mmol) and DIPEA (1.72 ml, 10.1 mmol, 5.0 eq) in DMF (60 ml) was added dropwise to a solution of HATU (2.3 g, 6.06 mmol, 3.0 eq) and DIPEA (5.15 ml, 30.3 mmol, 15.0 eq) in DMF (40 ml) at room temperature for 3 hours. Upon completion, monitored by LCMS, Ammonia 7N in methanol was added and stirred for 30 minutes. Solvent was removed and the product was purified by flash chromatography on silica gel using as eluents mixtures of dichloromethane: methanol (from 100:0 to 20:1). The product fractions were collected and the solvent was evaporated to dryness. The product pure was precipitated with acetonitrile and dried under vacuum to lead a pale solid in 463 mg (71% yield).

MH+: 324.2

Retention time: 2.107 min

Fusion point: >300 ° C.

### Example F106

Example F106 was prepared following general scheme 3 and more precisely a similar procedure than for the example F105.

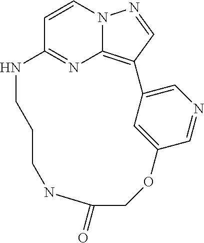 [SMILES: O=C1COc2cncc(c2)-c2cnn3ccc(nc23)NCCCN1]

Yield: 5 mg, 2.9%

MH+: 325.2

Retention time: 1.343 min

Melting point: ND

Preparation of Intermediate F107

Intermediate F107 was prepared following general scheme 3

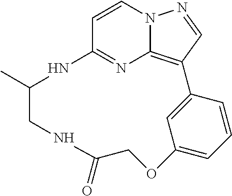 [SMILES: CC1CNC(=O)COc2cccc(c2)-c2cnn3ccc(nc23)N1]

The title compound was prepared following a similar procedure than for the example F129.

MH+: 324.2

### Example F108

Example F108 was prepared following general scheme 3

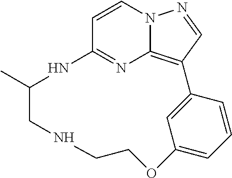 [SMILES: CC1CNCCOc2cccc(c2)-c2cnn3ccc(nc23)N1]

Step A

The intermediate F107 (163 mg, 0.5 mmol, 1.0 eq) was dissolved in Borane dimethylsulfide 2M in THF (0.38 ml, 5.0 mmol, 10.0 eq) and THF (1.5 ml), gas evolved. The reaction mixture was stirred for 32 h at rt. Upon completion, monitored by LCMS, the reaction mixture was quenched with 2N HCl and heated for 1 h at 100° C. (THF evaporated). The product was extracted with DCM 2× and once with DCM:MeOH 9:1. The combined organic layers were concentrated. The crude was purified by flash chromatography using a mixture of DCM:MeOH 98:2 to 95:5 slow. The compound was further purified by PREP HPLC to lead the title compound in 47 mg (30% yield).

MH+: 310.2

Retention time: 1.952

TABLE 1

| | |
|---|---|
| 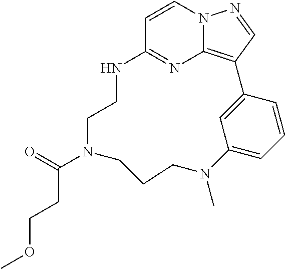 [SMILES: COCCC(=O)N1CCCN(C)c2cccc(c2)-c2cnn3ccc(nc23)NCC1] | Compound B19, Example B16 |
| 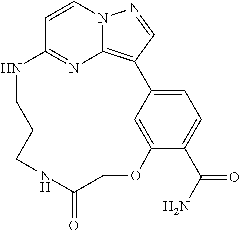 [SMILES: NC(=O)c1ccc2cc1OCC(=O)NCCCNc1ccn3ncc-2c3n1] | Compound B21, example B61 |
| 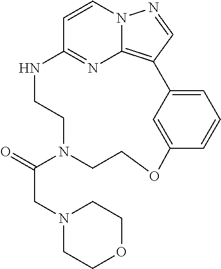 [SMILES: O=C(CN1CCOCC1)N1CCNc2ccn3ncc(c3n2)-c2cccc(c2)OCC1] | Compound B36, Example B23 |
| 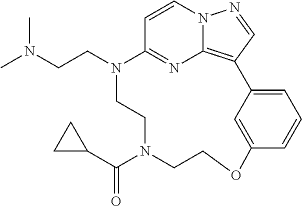 [SMILES: CN(C)CCN1CCN(C(=O)C2CC2)CCOc2cccc(c2)-c2cnn3ccc1nc23] | Compound B48, Example B35 |
| 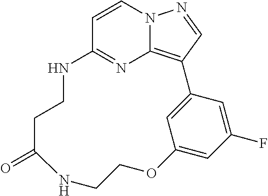 [SMILES: O=C1CCNc2ccn3ncc(c3n2)-c2cc(F)cc(c2)OCCN1] | Compound B74, Example B63 |
| 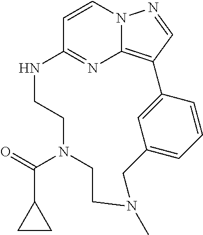 [SMILES: CN1CCN(C(=O)C2CC2)CCNc2ccn3ncc(c3n2)-c2cccc(c2)C1] | Compound B76, Example B65 |
| 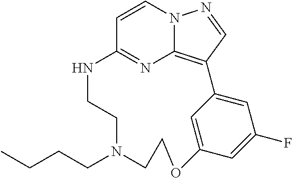 [SMILES: CCCCN1CCNc2ccn3ncc(c3n2)-c2cc(F)cc(c2)OCC1] | Compound F81, Example F81 |
| 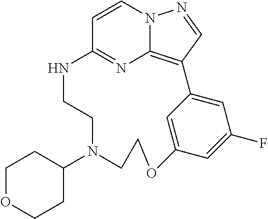 [SMILES: Fc1cc2cc(c1)-c1cnn3ccc(nc13)NCCN(C1CCOCC1)CCO2] | Compound F82, Example F82 |
| 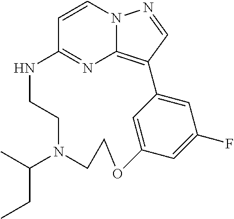 [SMILES: CCC(C)N1CCNc2ccn3ncc(c3n2)-c2cc(F)cc(c2)OCC1] | Compound F83, Example F83 |
| 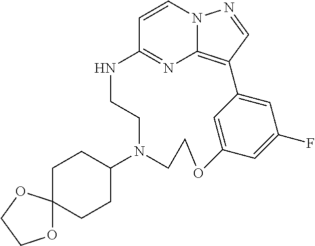 [SMILES: Fc1cc2cc(c1)-c1cnn3ccc(nc13)NCCN(C1CCC3(CC1)OCCO3)CCO2] | Compound F84, Example F84 |
| 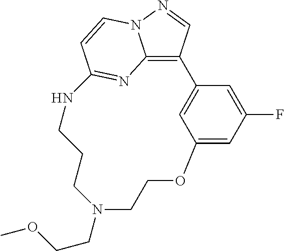 [SMILES: COCCN1CCCNc2ccn3ncc(c3n2)-c2cc(F)cc(c2)OCC1] | Compound F86, Example F86 |
| 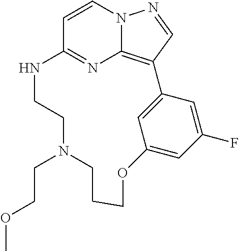 [SMILES: COCCN1CCCOc2cc(F)cc(c2)-c2cnn3ccc(nc23)NCC1] | Compound F87, Example F87 |
| 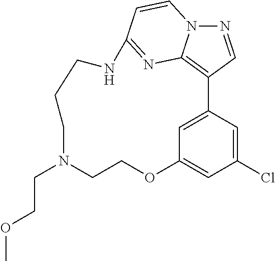 [SMILES: COCCN1CCCNc2ccn3ncc(c3n2)-c2cc(Cl)cc(c2)OCC1] | Compound F88, Example F88 |
| 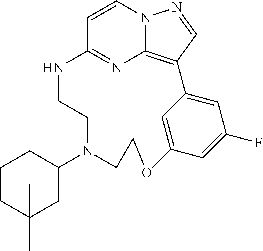 | Compound F89, Example F89 |
| 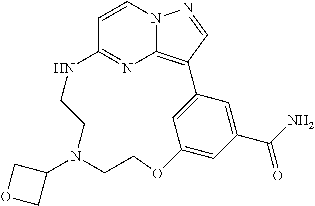 [SMILES: NC(=O)c1cc2cc(c1)-c1cnn3ccc(nc13)NCCN(C1COC1)CCO2] | Compound F91, Example F91 |
| 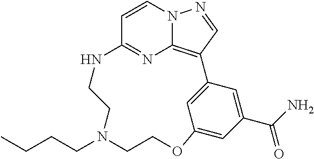 [SMILES: CCCCN1CCNc2ccn3ncc(c3n2)-c2cc(cc(C(N)=O)c2)OCC1] | Compound F92, Example F92 |
| 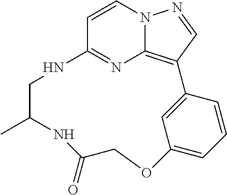 [SMILES: CC1CNc2ccn3ncc(c3n2)-c2cccc(c2)OCC(=O)N1] | Compound F105, Example F105 |
| 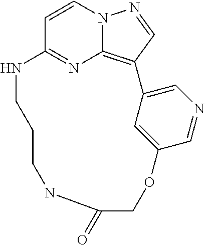 [SMILES: O=C1COc2cncc(c2)-c2cnn3ccc(nc23)NCCCN1] | Compound F106, Example F106 |
| 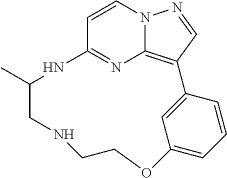 [SMILES: CC1CNCCOc2cccc(c2)-c2cnn3ccc(nc23)N1] | Compound F108, Example F108 |

The compounds were identified according to the analytical methods and the analytical results described in WO2013/045653 A1 and WO2013/046029 A1.

TABLE 2
Melting points

| COMPOUND | MELTING |
|---|---|
| N^{o} | POINT (° C.) |
| F81 | 370.2 |
| F82 | 398.4 |
| F83 | 370.4 |
| F84 | 454.3 |
| F86 | 386.2 |
| F87 | 386.2 |
| F88 | 402.1 |
| F89 | 423.3 |
| F91 | 395.2 |
| F92 | 395.2 |
| F105 | 324.2 |
| F106 | 325.2 |
| F108 | 310.2 |

TABLE 3
LCMS data

| COMPOUND | MASS (MH)⁺ | RETENTION | LCMS |
|---|---|---|---|
| NUMBER | PEAK | TIME (min) | METHOD |
| F81 | 370.2 | 2.549 | 2 |
| F82 | 398.4 | 2.297 | 2 |
| F83 | 370.4 | 2.385 | 2 |
| F84 | 454.3 | 2.410 | 2 |
| F86 | 386.2 | 2.083 | 2 |
| F87 | 386.2 | 2.131 | 2 |
| F88 | 402.1 | 2.252 | 2 |
| F89 | 423.3 | 2.942 | 2 |
| F91 | 395.2 | 1.789 | 2 |
| F92 | 395.2 | 1.633 | 2 |
| F105 | 324.2 | 2.107 | 2 |
| F106 | 325.2 | 1.343 | 2 |
| F108 | 310.2 | 1.952 | 2 |

The inhibition of RIP2 kinase was assessed using RIP2 recombinant protein in an in vitro peptide-based kinase assay.

B. Kinase Activity Assay

The inhibition of RIP2 kinase was assessed using RIP2 recombinant protein in an in vitro peptide-based kinase assay.

Protocol

A radiometric protein kinase assay (³³PanQinase® (³³PanQinase Activity Assay) is used for measuring the kinase activity. All assays are performed in 96-well FlashPlates™ from Perkin Elmer in a 50 μl reaction volume. The reaction cocktail is pipetted in 4 steps in the following order:

10 μl of non-radioactive ATP solution (in H₂O)

25 μl of assay buffer/[γ-³³P]-ATP mixture

5 μl of test sample in 10% DMSO

10 μl of enzyme/substrate mixture

The assay for RIP2 contains 70 mM HEPES-NaOH pH 7.5, 3 mM MgCl₂, 3 mM MnCl₂, 3 μM Na-orthovanadate, 1.2 mM DTT, 50 μg/ml PEG20000, ATP (3.0 μM), [γ-³³P]-ATP (approx. 5×10⁰⁵ cpm per well), protein kinase RIP2 (15.7 nM) and substrate (RBER-Chktide), 2.0 μg/50 μl). The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. Incorporation of ³³Pi (counting of “cpm”) was determined with a microplate scintillation counter.

Compounds

The compounds are dissolved to 10 mM in DMSO. Where needed, solutions are sonicated in a bath sonicator.

Table 4 provides the pIC₅₀ values and % Remaining activity values at two concentrations (1 μM and 0.1 μM) of the compounds according to the invention, obtained using the above mentioned kinase assay.

TABLE 4

| | | % Remaining | % Remaining |
|---|---|---|---|
| | | RIP2 activity at | RIP2 activity at |
| Compound N^{o} | IC₅₀ for RIP2 | 1 μM | 0.1 μM |
| B19 | | ** | ** |
| B21 | +++ | ** | ** |
| B36 | +++ | ** | ** |
| B48 | +++ | ** | ** |
| B74 | ++ | ** | ** |
| B76 | ++ | ** | * |
| F81 | +++ | ** | ** |
| F82 | +++ | ** | ** |
| F83 | +++ | ** | ** |
| F84 | ++ | ** | ** |
| F86 | +++ | ** | ** |
| F87 | +++ | ** | ** |
| F88 | +++ | ** | ** |
| F89 | ++ | ** | ** |
| F91 | +++ | ** | ** |
| F92 | +++ | ** | ** |
| F105 | ++ | ** | * |
| F106 | ++ | ND | ND |
| F108 | ++ | ND | ND |

+ indicates an IC50 >1 μM,
++ indicates an IC50 of between 100 nM and 1 μM, and
+++ indicates an IC50 <100 nM
* indicates a % remaining kinase activity above 50%,
** indicates a % remaining kinase activity below 50%
ND = Not determined

1. A method for the prevention and/or treatment of a RIP2-kinase associated disease; said method comprising administering to a subject in need thereof a compound of Formula I or a stereoisomer, tautomer, racemic, metabolite, pro- or predrug, salt, hydrate, N-oxide form, or solvate thereof, 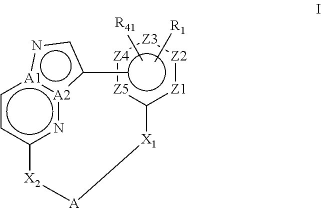
wherein:
A₁ and A₂ are selected from C and N; wherein when A₁ is C, then A₂ is N; and wherein when A₂ is C, then A₁ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —O—C₁₋₆alkyl, and —S—C₁₋₆alkyl;
R₂ is selected from —H, -halo, —OH, —C₁₋₆ alkyl, —O—C₁₋₆alkyl, —S—C₁₋₆alkyl, —(C═O)—C₁₋₆ alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, (C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃ , and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆ alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆ alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃ (C═O)—NR₂₅ R₂₆, —(C═S)—NR₂₅R₂₆, and —NR₃₃(C═S)—NR₂₅R₂₆; and
wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O) —NR₅₃ R₅₄, —NR₅₅ (C═O)—NR₅₃R₅₄, —(C═S)—NR₅₃ R₅₄ , and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C_{1 -6}alkyl, —C₃₋₆cyclo alkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆ alkyl, —O—C_{1 -6}alkyl, —S—C_{1 -6}alkyl, —NR₄₆R₄₇, —C₃₋₆cycloalkyl, —Ar₉ and -Het₈;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C_{1 -6} alkyl-, —NR₃—SO₂—, —NR₃—(C═O)—C₁₋₆alkyl-, —(C═O)—NR₃—C_{1 -6} alkyl-, —O—C₁₋₆alkyl-O—C₁₋₆alkyl- and —C_{1 -6} alkyl-NR₃—C₁₋₆alkyl-;
wherein each of said —C₁₋₆alkyl- is optionally and independently substituted with from 1 to 3 substituents selected from -halo, —OH, —C_{1 -6}alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆ alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4.

2. The method according to claim 1, wherein:
A₁ is C and A₂ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆ alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C═O)-Het₃, —(C═S)-Het₃, —(C═O)-Ar₂, —(C═S)-Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl-Het₂, —Ar₃, —(C—O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₂, —Ar₃, and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆alkyl, —O—(C═O)—C₁₋₆ alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C—S)—NR₃₁—(C═S)—R₃₂, -Het₆, —Ar₆, and —C₃₋₆cycloalkyl;
wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃(C═O)—NR₂₅ R₂₆, —(C═S)—NR₂₅R₂₆, and —NR₃₃(C═S)—NR₂₅R₂₆; and
wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O)—NR₅₃R₅₄, —NR₅₅(C═O)—NR₅₃R₅₄, —(C═S)—NR₅₃R₅₄, and —NR₅₅(C═S)—NR₅₃R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)⁻0⁻R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, -S —C₁₋₆alkyl, —C₃₋₆cyclo alkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C_{1 -6}alkyl-O—C₁₋₆alkyl- and —C_{1 -6}alkyl-NR₃—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4.

3. The method according to claim 1, wherein:
A₁ is N and A₂ is C
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —CN, —NR₉—SO₂R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from —H, -halo, —OH, —C₁₋₆alkyl, —O—C₁₋₆alkyl, —S—C₁₋₆alkyl, —(C═O)—C₁₋₆ alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, —Ar₂, —(C═O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃, —Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl -Het₂, —Ar₃, —(C—O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —C₃₋₆cycloalkyl, —Ar₃, and —NR₁₅R₁₆;
R₄ is independently selected from -halo, —OH, —C₁₋₆alkyl, —O—C₁₋₆ alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆ alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆, —NR₂₅R₂₆, —(C═O)—NR₂₅R₂₆, —NR₃₃ (C═O)—NR₂₅R₂₆, —(C═S)—NR₂₅R₂₆, and —NR₃₃(C═S)—NR₂₅R₂₆; and
wherein each of said —C₃₋₆cycloalkyl is optionally and independently substituted with from 1 to 3 substituents selected from —C₁₋₆alkyl, ═O, -halo, —OH, —O—C₁₋₆alkyl, —S—C₁₋₆alkyl, -Het₁₂, —Ar₁₁, and —NR₅₃R₅₄, —(C═O) —NR₅₃R₅₄, —NR₅₅(C═O)—NR₅₃ R₅₄, —(C═S) —NR₅₃R₅₄, and —NR₅₅(C═S)—NR₅₃R₅₄;
R₈ is selected from —NR₃₄—(C—O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R_{I4}, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S —C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;
R₄₃ is selected from —H —C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C_{1 -6} alkyl-, —NR₃—SO₂—, —NR₃—(C═O)—C₁₋₆alkyl-, —(C═O)—NR₃—C_{1 -6} alkyl-, —O—C₁₋₆alkyl-O—C₁₋₆alkyl- and —C_{1 -6} alkyl-NR₃—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C═O)—NR₂—C₁₋₆alkyl-, —O—C₁₋₆alkyl-O—C₁₋₆alkyl- and —C₁₋₆alkyl-NR₂—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉, and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉, Het₁₀, and Het₁₂ is optionally and independently substituted with from 1 to 3 substituents selected from -halo, —OH, C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4.

4. The method according to claim 1,
wherein:
A₁ and A₂ are selected from C and N; wherein when A₁ is C, then A₂ is N; and wherein when A₂ is C, then A₁ is N;
R₁ and R₄₁ are each independently selected from —H, -halo, —C₁₋₆alkyl, —(C═O)—R₄, and —CN;
wherein each of said —C₁₋₆alkyl is optionally and independently substituted with from 1 to 3 substituents selected from —O—C₁₋₆alkyl;
R₂ is selected from H, and —C₁₋₆alkyl; wherein each of said —C₁₋₆alkyl is optionally and independently substituted with —NR₁₃R₁₄;
R₃ is selected from H, and —C₁₋₆alkyl; wherein each of said —C₁₋₆alkyl is optionally and independently substituted with —NR₁₅R₁₆;
R₄ is —NR₁₇R₁₈;
R₅ is H;
R₆ is selected from —C₁₋₆alkyl, —(C═O)—C₁₋₆alkyl, —(C═O)—C₃₋₆cycloalkyl, -Het₆, and —C₃₋₆cycloalkyl;
wherein each of said —C₁₋₆alkyl is optionally and independently substituted with from 1 to 3 substituents selected from —O—C₁₋₆alkyl and -Het₆;
and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from H, and —C₁₋₆alkyl;
A is selected from —(CH₂)ₙ—Y—(CH₂)ₘ—, —NR₆—, and —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4.

5. The method according to claim 1, wherein:
A₁ is C and A₂ is N;
R₁ and R₄₁ are each independently selected from —H, -halo, —C₁₋₆alkyl, —(C═O)—R₄, and —CN;
wherein each of said —C₁₋₆alkyl is optionally and independently substituted with from 1 to 3 substituents selected from —O—C₁₋₆alkyl;
R₂ is selected from H, and —C₁₋₆alkyl; wherein each of said —C₁₋₆alkyl is optionally and independently substituted with —NR₁₃R₁₄;
R₃ is selected from H, and —C₁₋₆alkyl; wherein each of said —C₁₋₆alkyl is optionally and independently substituted with —NR₁₅R₁₆;
R₄ is —NR₁₇R₁₈;
R₅ is H;
R₆ is selected from —C₁₋₆alkyl, —(C═O)—C₁₋₆alkyl, —(C═O)—C₃₋₆cycloalkyl, -Het₆, and —C₃₋₆cycloalkyl;
wherein each of said —C₁₋₆alkyl is optionally and independently substituted with from 1 to 3 substituents selected from —O—C₁₋₆alkyl and -Het₆;
and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from H, and —C₁₋₆alkyl;
R₄₃ is selected from —H, and —C₁₋₆alkyl;
A is selected from —(CH₂)ₙ—Y—(CH₂)ₘ—, —NR₆—, and —(C═O)—NR₅—;
X₁ is selected from O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4.

6. The method according to claim 1,
wherein:
A₁ is N and A₂ is C;
R₁ and R₄₁ are each independently selected from H, -halo, —C₁₋₆alkyl, —(C═O)—R₄, and —CN;
wherein each of said —C₁₋₆alkyl is optionally and independently substituted with from 1 to 3 substituents selected from —O—C₁₋₆alkyl;
R₂ is selected from —H, and —C₁₋₆alkyl; wherein each of said —C₁₋₆alkyl is optionally and independently substituted with —NR₁₃R₁₄;
R₃ is selected from —H, and —C₁₋₆alkyl; wherein each of said —C₁₋₆alkyl is optionally and independently substituted with —NR₁₅R₁₆;
R₄ is —NR₁₇R₁₈;
R₅ is —H;
R₆ is selected from —C₁₋₆alkyl, —(C═O)—C₁₋₆alkyl, —(C═O)—C₃₋₆cycloalkyl, -Het₆, and —C₃₋₆cycloalkyl;
wherein each of said —C₁₋₆alkyl is optionally and independently substituted with from 1 to 3 substituents selected from —O—C₁₋₆alkyl and -Het₆;
and wherein each of said —C₃₋₆cycloalkyl is optionally and independently substituted with from 1 to 3 substituents selected from —C₁₋₆alkyl;
R₁₃, R₁₄, R₁₅, R₁₆, R₁₇, R₁₈, are each independently selected from —H, and —C₁₋₆alkyl;
R₄₃ is selected from —H, and —C₁₋₆alkyl;
A is selected from —(CH₂)ₙ—Y—(CH₂)ₘ—, —NR₆—, and —(C═O)—NR₅—;
X₁ is selected from —O—C₁₋₆alkyl-, —C₁₋₆alkyl-NR₃—, and —C₁₋₆alkyl-NR₃—C₁₋₆alkyl-; wherein each of said —C₁₋₆alkyl- is optionally and independently substituted with from 1 to 3 substituents selected from —C₁₋₆alkyl;
X₂ is selected from —O—C₁₋₆alkyl-, —C₁₋₆alkyl-NR₂—; wherein each of said —C₁₋₆alkyl- is optionally and independently substituted with from 1 to 3 substituents selected from —C₁₋₆alkyl;
Y is —NR₄₃—;
Het₆ is a 4- to 10-membered heterocycle having from 1 to 3 heteroatoms selected from O, N and S;
Z₁, Z₂, Z₃, Z₄ and Z₅ are each independently selected from C and N; and
m and n are each independently 1, 2, 3, or 4.

7. The method according to claim 1, wherein the pyrazolopyrimidine or the imidazopyridazine moiety is linked to the aryl or heteroaryl moiety at position Z₄ or Z₅, in accordance with the numbering as provided in Formula I.

8. The method according to claim 1, wherein R₁ is linked to the aryl or heteroaryl moiety at position Z₁, Z₂ or Z₃, in accordance with the numbering as provided in Formula I.

9. A compound selected from the group consisting of:

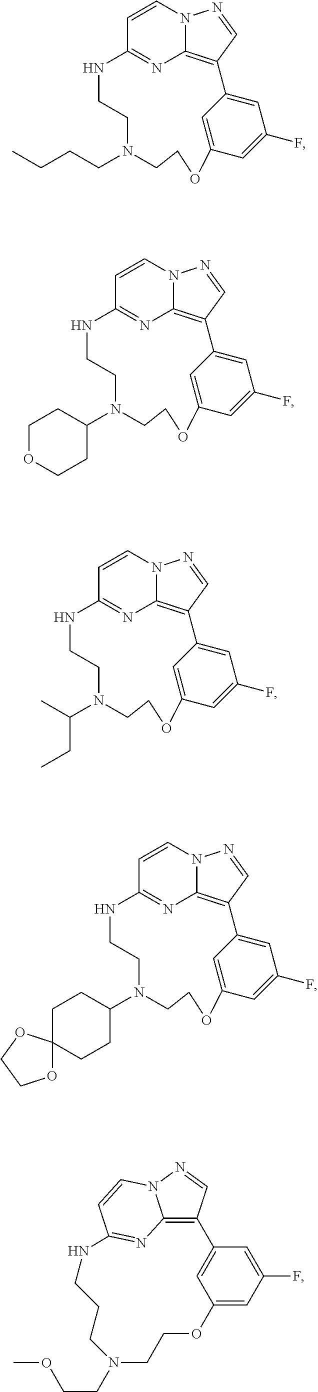 [SMILES: CCCCN1CCNc2ccn3ncc(c3n2)-c2cc(F)cc(c2)OCC1 ; Fc1cc2cc(c1)-c1cnn3ccc(nc13)NCCN(C1CCOCC1)CCO2 ; CCC(C)N1CCNc2ccn3ncc(c3n2)-c2cc(F)cc(c2)OCC1 ; Fc1cc2cc(c1)-c1cnn3ccc(nc13)NCCN(C1CCC3(CC1)OCCO3)CCO2 ; COCCN1CCCNc2ccn3ncc(c3n2)-c2cc(F)cc(c2)OCC1]

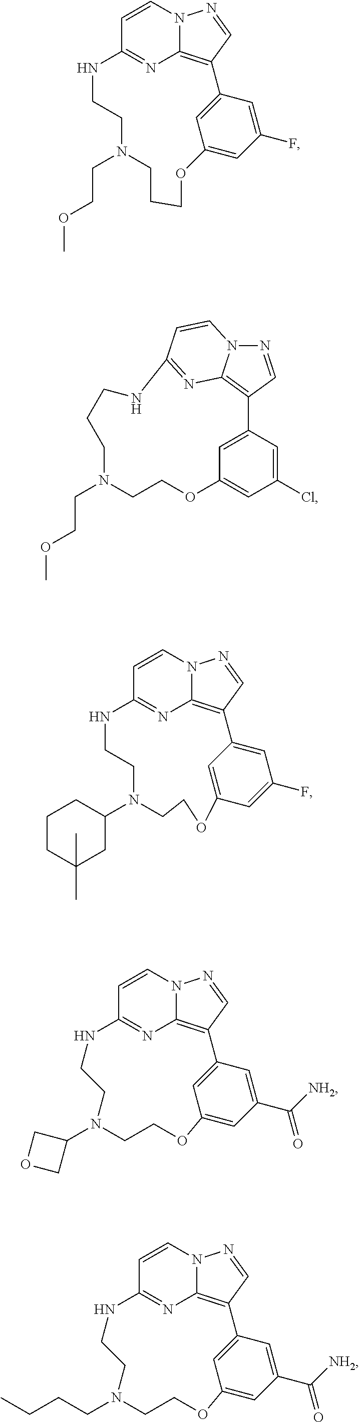

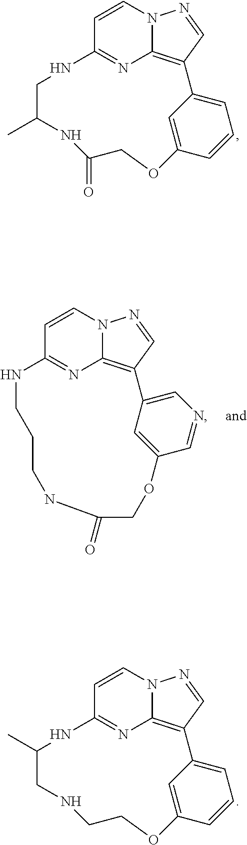 [SMILES: CC1CNc2ccn3ncc(c3n2)-c2cccc(c2)OCC(=O)N1 ; O=C1COc2cncc(c2)-c2cnn3ccc(nc23)NCCCN1 ; CC1CNCCOc2cccc(c2)-c2cnn3ccc(nc23)N1]

10. (canceled)

11. A pharmaceutical composition for the prevention and/or treatment of a RIP2-kinase associated disease comprising:
the compound according to claim 9: and
at least one pharmaceutically acceptable carrier, diluent, or excipient.

12. An in vitro method of inhibiting activity of RIP2 kinase, the method comprising contacting RIP2 kinase with 1 a compound of Formula I or a stereoisomer, tautomer, racemic, metabolite, pro- or predrug, salt, hydrate, N-oxide form, or solvate thereof, 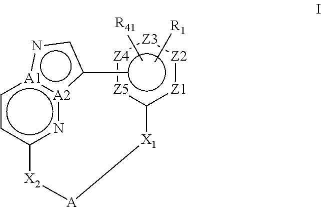
wherein:
A₁ and A₂ are selected from C and N; wherein when A₁ is C, then A₂ is N; and wherein when A₂ is C, then A₁ is N;
R₁ and R₄₁ are each independently selected from —H, -halo, —OH, —C₁₋₆alkyl, —O—C₁₋₆alkyl, —S—C₁₋₆alkyl, —NR₉R₁₀, —(C═O)—R₄, —(C═S)—R₄, —SO₂—R₄, —CN, —NR₉—SO₂—R₄, —C₃₋₆cycloalkyl, —Ar₇ and -Het₁; wherein each of said —C₁₋₆alkyl is optionally and independently substituted with from 1 to 3 substituents selected from -halo, —OH, —NR₁₁R₁₂, —O—C₁₋₆alkyl, and —S—C₁₋₆alkyl;
R₂ is selected from H, -halo, —OH, —C₁₋₆alkyl, —O—C_{1 -6}alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₇R₂₈, —(C═S)—NR₂₇R₂₈, —C₃₋₆cycloalkyl, -Het₃, —Ar₂, —(C—O)-Het₃, —(C═S)-Het₃, —(C═O)—Ar₂, —(C═S)—Ar₂, —(C═O)—C₃₋₆cycloalkyl, —(C═S)—C₃₋₆cycloalkyl, and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, -Het₃—Ar₂, and —NR₁₃R₁₄;
R₃ is selected from —H, -halo, —OH, —C₁₋₆alkyl, —O—C₁₋₆alkyl, —S—C₁₋₆alkyl, —(C═O)—C₁₋₆alkyl, —(C═S)—C₁₋₆alkyl, —(C═O)—O—C₁₋₆alkyl, —(C═S)—O—C₁₋₆alkyl, —(C═O)—NR₂₉R₃₀, —(C═S)—NR₂₉R₃₀, —C₃₋₆cycloalkyl-Het₂, —Ar₃, —(C═O)-Het₂, —(C═S)-Het₂, —(C═O)—Ar₃, —(C═S)—Ar₃, —(C═O)—C₃₋₆cycloalkyl, —(C═S)—C₃₋₆cycloalkyl and —SO₂—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cyclo alkyl, -Het₂, —Ar₃ , and —NR₁₅R₁₆;
R₄ is independently selected from -halo, —OH, —C₁₋₆alkyl, —O—C₁₋₆alkyl, —S—C₁₋₆alkyl, —NR₁₇R₁₈, —C₃₋₆cycloalkyl, —Ar₈ and -Het₄;
R₅ and R₇ are each independently selected from —H, —OH, -halo, —C₁₋₆alkyl, —O—C₁₋₆alkyl, —S—C₁₋₆alkyl, -Het₉, —Ar₁, —C₃₋₆cycloalkyl, —SO₂—Ar₁, —SO₂, —SO₂—C₁₋₆alkyl, —(C═O), —(C═O)—C₁₋₆alkyl, —(C═S), —(C═S)—C₁₋₆ alkyl, —O—(C═O)—C₁₋₆alkyl, —O—(C═S)—C₁₋₆alkyl, —(C═O)—O—C₁₋₆alkyl, and —(C═S)—O—C₁₋₆alkyl; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, —Ar₁, -Het₉, and —NR₂₃R₂₄;
R₆ is selected from —C₁₋₆alkyl, —SO₂, —SO₂—C₁₋₆alkyl, —SO₂—C₃₋₆cycloalkyl, —(C═O), —(C═O)—C₁₋₆alkyl, —(C═O)—C₂₋₆alkenyl, —(C═O)—O—C₁₋₆alkyl, —(C═O)-Het₆, —(C═O)—Ar₆, —(C═O)—C₃₋₆cycloalkyl, —(C═O)—NR₃₁R₃₂, —(C═O)—NR₃₁—(C═O)—R₃₂, —(C═S), —(C═S)—C₁₋₆alkyl, —(C═S)—C₂₋₆alkenyl, —(C═S)—O—C₁₋₆alkyl, —(C═S)-Het₆, —(C═S)—Ar₆, —(C═S)—C₃₋₆cycloalkyl, —(C═S)—NR₃₁R₃₂, —(C═S)—NR₃₁—(C═S)—R₃₂, -Het₆, —Ar₆, and —C₃₋₆cycloalkyl;
wherein each of said —C₁₋₆alkyl is optionally and independently substituted with from 1 to 3 substituents selected from ═O, -halo, —OH, —O—C₁₋₆alkyl, —S—C₁₋₆alkyl, —C₃₋₆cycloalkyl, -Het₆, —Ar₆—, NR₂₅R₂₆, —(C═O)—NR₂₅R₂₆, —NR₃₃(C═O)—NR₂₅ R₂₆, —(C═S)—NR₂₅R₂₆, and —NR₃₃(C═S)—NR₂₅R₂₆; and
wherein each of said —C₃₋₆cycloalkyl is optionally and independently substituted with from 1 to 3 substituents selected from —C₁₋₆ alkyl, ═O, -halo, —OH, —O—C₁₋₆alkyl, —S—C₁₋₆alkyl, -Het₁₂, —Ar₁₁, and —NR₅₃R₅₄, —(C═O) —NR₅₃R₅₄, —NR₅₅(C═O)—NR₅₃R₅₄, —(C═S)—NR₅₃R₅₄ and —NR₅₅(C═S)—NR₅₃ R₅₄;
R₈ is selected from —NR₃₄—(C═O)—R₃₅, —NR₃₄—(C═S)—R₃₅, —NR₃₆—(C═O)—NR₃₄R₃₅, —NR₃₆—(C═S)—NR₃₄R₃₅, —NR₃₄—(SO₂)—R₃₅, —NR₃₄—(C═O)—O—R₃₅, —NR₃₄—(C═S)—O—R₃₅, —O—(C═O)—NR₃₄R₃₅, and —O—(C═S)—NR₃₄R₃₅;
R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R₂₉, R₃₀, R₃₁, R₃₂, R₃₃, R₃₄, R₃₅, R₃₆, R₃₇, R₃₈, R₃₉, R₄₀, R₄₄, R₄₅, R₄₆, R₄₇, R₄₈, R₄₉, R₅₀, R₅₃, R₅₄ and R₅₅ are each independently selected from —H, -halo, ═O, —OH, —C₁₋₆alkyl, —O—C₁₋₆alkyl, —S—C₁₋₆alkyl, —C₃₋₆cycloalkyl, —Ar₅ and -Het₇; wherein each of said —C₁₋₆alkyl is optionally and independently substituted with from 1 to 3 substituents selected from -halo, —OH, —O—C₁₋₆alkyl, —S—C₁₋₆alkyl, —C₃₋₆cycloalkyl, -Het₇, —Ar₅ and —NR₅₁R₅₂;
R₅₁ and R₅₂ are each independently selected from —H, -halo, —OH, —C₁₋₆alkyl, —O—C₁₋₆alkyl, —S—C₁₋₆alkyl, —C₃₋₆cycloalkyl, —Ar₁₀ and -Het₁₀;
R₄₂ is selected from —H, —OH, -halo, —C₁₋₆alkyl, —O—C₁₋₆alkyl, —S—C₁₋₆alkyl, —NR₄₆R₄₇, —C₃₋₆cycloalkyl, —Ar₉ and -Het₈;.
R₄₃ is selected from —H—C₁₋₆alkyl, and —C₃₋₆cycloalkyl; wherein each of said —C₁₋₆alkyl is optionally and independently substituted with from 1 to 3 substituents selected from -halo, —OH, —O—C₁₋₆alkyl, —S—C₁₋₆alkyl, -Het₅, —C₃₋₆cycloalkyl —Ar₄, and —NR₄₄R₄₅;
A is selected from —(CH₂)ₙ—Y—(CH₂)ₘ—, —(C═O)—, —(C═S)—, —(C═N)—R₄₉—, —(SO₂)—, —SO₂—NR₅—, —(C═O)—NR₅—, —(C═S)—NR₅—, —NR₅—(C═O)—NR₇—, —NR₅—(C═S)—NR₇—, —NR₆,—, —NR₅—(C═O)—O—, —NR₅—(C═S)—O—, and —CHR₈—;
X₁ is selected from —C₁₋₆alkyl-, —O—C₁₋₆alkyl-, —S—C₁₋₆alkyl-, —(C═O)—, —NR₃—(C═O)—, —C₁₋₆alkyl-NR₃—, —NR₃—, —(C═O)—, —NR₃—(C═O)—NR₄₈—, —NR₃—C₁₋₆alkyl-, —NR₃—SO₂—, —NR₃—(C═O)—C₁₋₆alkyl-, —(C═O)—NR₃—C₁₋₆alkyl-, —O—C₁₋₆alkyl-O—C₁₋₆alkyl- and —C₁₋₆alkyl-NR₃—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl, and —NR₃₇R₃₈;
X₂ is selected from —C₁₋₆alkyl-, —O—C₁₋₆alkyl-, —S—C₁₋₆alkyl-, —(C═O)—, —NR₂—(C═O)—, —C₁₋₆alkyl-NR₂—, —NR₂—, —(C═O)—, —NR₂—(C═O)—NR₅₀—, —NR₂—C₁₋₆alkyl-, —NR₂—SO₂—, —NR₂—(C═O)—C₁₋₆alkyl-, —(C—O)—NR₂—C₁₋₆alkyl-, —O—C₁₋₆alkyl-O—C₁₋₆alkyl- and —C₁₋₆alkyl-NR₂—C₁₋₆alkyl-;
wherein each of said —C₁₋₆alkyl- is optionally and independently substituted with from 1 to 3 substituents selected from -halo, —OH, —C₁₋₆alkyl, —O—C₁₋₆alkyl, —S—C₁₋₆alkyl, -phenyl and —NR₃₉R₄₀;
Y is selected from a direct bond, —CHR₄₂—, —O—, —S—, and —NR₄₃—;
Ar₁, Ar₂, Ar₃, Ar₄, Ar₅, Ar₆, Ar₇, Ar₈, Ar₉, Ar₁₀ and Ar₁₁ are each independently a 5- to 10-membered aromatic heterocycle optionally comprising 1 or 2 heteroatoms selected from O, N and S; each of said Ar₁, Ar₂, Ar₃, Ar₄, Ar₅, Ar₆, Ar₇, Ar₈, Ar₉ and Ar₁₀ being optionally and independently substituted with from 1 to 3 substituents selected from -halo, —OH, —C₁₋₆alkyl, —O—C₁₋₆alkyl, —S—C₁₋₆alkyl, and —NR₁₉R₂₀; wherein each of said —C₁₋₆alkyl is optionally and independently substituted with from 1 to 3-halo;
Het₁, Het₂, Het₃, Het₄, Het₅, Het₆, Het₇, Het₈, Het₉, Het₁₀, and Het₁₂ are each independently a 4- to 10-membered heterocycle having from 1 to 3 heteroatoms selected from O, N and S, wherein each of said Het₁, Het₂, Het₃, Het₄, Het₅, Het₆, Het₇, Het₈, Het₉. Het₁₀, and Het₁₂ is optionally and independently substituted with from 1 to 3 substituents selected from -halo, —OH, —C₁₋₆alkyl, —OC₁₋₆alkyl, —SC₁₋₆alkyl, ═O, —(C═O)—C₁₋₆alkyl, and —NR₂₁R₂₂; wherein each of said —C₁₋₆alkyl is optionally and independently substituted with from 1 to 3-halo;
Z₁, Z₂, Z₃, Z₄ and Z₅ are each independently selected from C and N; and
m and n are each independently 1, 2, 3, or 4.

13. (canceled)

14. (canceled)

15. The method according to claim 1, wherein the RIP2-kinase associated disease is an inflammatory disorder selected from the group consisting of Crohn's disease, bowel disease, Sarcoidosis, psoriasis, rheumatoid arthritis, asthma, ulcerative colitis, lupus, uveitis, blau syndrome, granulomatous inflammation, behcee s disease, multiple sclerosis and insulin-resistant type 2 diabetes.
